(19) United States
(12) Patent Application Publication
(10) Pub. No.: US 2019/0297960 A1
(43) Pub. Date: Oct. 3, 2019

(54) METHODS AND COMPOSITIONS FOR WEARABLE TEXTILE ELECTRONIC DEVICES

(71) Applicant: North Carolina State University, Raleigh, NC (US)

(72) Inventors: JESSE S. JUR, Raleigh, NC (US); HASAN SHAHARIAR, Raleigh, NC (US); RAJ BHAKTA, Raleigh, NC (US)

(21) Appl. No.: 16/285,854

(22) Filed: Feb. 26, 2019

Related U.S. Application Data
(60) Provisional application No. 62/635,540, filed on Feb. 26, 2018.

(51) Int. Cl. A41D 1/00; A61F 7/02; A42B 1/24; A42B 3/04; A41B 11/00; A41D 19/00; A41D 13/015; A61F 13/00; A61B 5/00; B41M 3/00; B41M 5/00; D06P 5/00; D06P 5/30; C09D 11/52; C09D 11/322; C09D 11/037; C09D 11/10

(52) U.S. Cl. CPC A41D 1/002; A61F 2007/0071; A42B 1/242; A42B 3/0433; A41B 11/00; A41D 19/0027; A41D 13/015; A61F 13/00051; A61B 5/6812; A61B 5/6806; A61B 5/6807; A61B 5/6803; B41M 3/006; B41M 5/0047; D06P 5/001; D06P 5/30; C09D 11/52; C09D 11/322; C09D 11/037; C09D 11/10; A61F 7/02

(57) ABSTRACT

In one aspect, the disclosure relates to methods for on-demand ink deposition processes for printing conductive inks on textiles. The disclosed methods can be used to fabricate various disclosed wearable textile electronic devices comprising a textile product, such as a textile garment, and one or more electronic component such as a vertical interconnect access device, resistive printed heater, and a meshed-patch antenna. This abstract is intended as a scanning tool for purposes of searching in the particular art and is not intended to be limiting of the present disclosure.

### CROSS-REFERENCE TO RELATED APPLICATIONS

This application claims the benefit of and priority to co-pending U.S. Provisional Patent Application No. 62/635,540, filed on Feb. 26, 2018, entitled “Methods and Compositions for Wearable Textile Electronic Devices,” the contents of which is incorporated by reference herein in its entirety.

### STATEMENT REGARDING FEDERALLY SPONSORED RESEARCH

This invention was made with government support under grant number 1160483 awarded by the National Science Foundation. The government has certain rights in the invention.

### BACKGROUND

Wearable textile electronic devices, that is, textiles comprising conductive and electronic components have widespread application and use in diverse fields such as healthcare, fitness, sensors, and energy harvesting. The vast potential of electronic textiles remains to be tapped due to a variety of production issues that currently limit widespread commercialization of textiles comprising conductive and electronic components.

Direct-write printing is a promising method for printing continuous roll-to-roll large-area electronics directly onto flexible substrates with a one-step process, ruling out the need for complex and materials-intensive lithographic processes. However, direct-write printing conductive circuits on rough textile materials is severely limited due to textile's inherent high surface roughness and porosity and the industry has lacked commercially viable high-throughput methods that meet processing criteria for the textile electronics industry.

Despite advances in research directed to methods and compositions for wearable textile electronic devices, there remain a scarcity of commercially viable and scalable methods and compositions. These needs and other needs are satisfied by the present disclosure.

### SUMMARY

In accordance with the purpose(s) of the disclosure, as embodied and broadly described herein, the disclosure, in one aspect, relates to direct-write methods utilizing a drop on demand printhead dispensing at least one conductive ink.

Disclosed are methods for forming a conductive material comprising applying a conductive ink with a printer to a substrate material; wherein the conductive ink comprises a conductive microparticle; wherein the conductive ink has a viscosity of about 10,0001 cps to about 30100,000+ cps when determined at a 1 s⁻¹ shear rate; wherein the printer comprises a drop on demand ink jet printhead comprising at least one nozzle; wherein the at least one nozzle tip is at a distance of about 0.1 mm to about 0.4 mm from the substrate material; wherein the conductive ink is dispensed from the at least one nozzle at a dispensing velocity of about 50 mm/s to about 200 mm/s; and wherein the conductive ink is dispensed from the at least one nozzle at a fluid pressure of about 1 psi to about 100 psi.

Also disclosed are articles comprising a component made by a disclosed method. In some aspects, the component can be a printed resistive heating device, a printed antenna, a vertical interconnect access, or combinations thereof. In various aspects, the article can be a garment, an article of apparel, an article of footwear, an article of protective clothing, a helmet, a hat, a sock, a glove, a ballistic material, or an article of body armor. In a further aspect, the article can be a medical device, a wound covering, a wound dressing, a medical mesh, or a medical fabric.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the disclosure.

FIGS. 1A-1H shows representative aspects of disclosed direct write devices and processes. FIG. 1A shows a schematic representation of a disclosed direct-write process with a drop-on-demand mode being used to print on a textile substrate. FIG. 1B shows a representative wearable textile electronic device, a meshed-patch antenna device, being printed using this a direct-write drop-on-demand mode, similar to that shown in FIG. 1A, being directly printed on a nonwoven textile. FIG. 1C shows a representative photomicrograph of the nonwoven textile of FIG. 1B comprising the printed electronic device shown in a cross-section at the interface between the printed electronic device and the nonwoven textile. FIG. 1D shows an alternative aspect of the disclosed direct write devices and processes. Specifically, FIG. 1D shows a schematic representation of a disclosed process comprising direct-write printing on a film such as thermoplastic polyurethane (TPU) heat-laminated onto a polyester-spandex knitted textile. FIG. 1E shows a representative wearable textile electronic device made using the process shown in FIG. 1D. Specifically, FIG. 1E shows a representative heat laminated interconnect device in a meandering pattern. FIG. 1F shows a representative schematic image of a wearable textile electronic device made using a disclosed direct-write process, such that the wearable textile electronic device comprises components for multi-modal sensing and/or energy harvesting in a garment. FIG. 1G shows a representative disclosed wearable textile electronic device, a electrocardiogram shirt, comprising a representative heat laminated interconnect device in a meandering pattern that has use as a wearable device for health monitoring. FIG. 1H shows an alternative wearable textile electronic device comprising heat laminated interconnect devices thereon, which an alternative aspect of a heat laminated interconnect device shown in FIG. 1E and made using a disclosed method such as that shown in FIG. 1D.

FIGS. 2A-2B show representative data for representative disclosed inks used in the disclosed methods. FIG. 2A shows representative data for viscosity versus shear rate of inks prepared with three different viscosities. As indicated in the figure, the disclosed Ag/AgCl inks are designated as having a certain viscosity 0/10, 0.5/10 and 1/10, which refers to the amount of diluent used to dilute 10 gm of the native ink, respectively, i.e., dilution with 0 ml, 0.5 ml and 1.0 ml of diluent, 2-butoxyethyl acetate. The data show that as the concentration of solvent increases, the viscosity decreases showing the shear thinning behavior of the ink. FIG. 2B shows representative data for elastic modulus versus shear stress of the inks described in FIG. 2A. The dashed lines for each ink intersect at a shear stress critical value termed as yield stress for each ink tested. Below the yield stress point, the elastic modulus of the ink does not change with shearing and acts like a solid material. A minimum yield stress must be exerted to allow for the non-newtonian thixotropic conductive inks to flow and thus allow for printability.

FIGS. 3A-3G show representative images pertaining to the flowability and viscoelastic behavior of a droplet of a disclosed ink on a textile substrate. FIG. 3A shows time lapse, chronological progression showing a needle tip and ejection of a droplet of a disclosed Ag/AgCl ink (viscosity 0/10) on a surface of a nonwoven textile, Evolon®. As indicated in the image, the needle tip to fabric surface gap was selected as 0.3 mm. As shown in the image, at this gap, the ink retains its shape after the shear stress and shear rate drops to zero following ejection from the nozzle. FIGS. 3B and 3C show images for the contact angle for an ink droplet at zero time and one minute after the ink droplet was deposited on an Evolon® nonwoven textile. The ink and injection conditions were the same as used for FIG. 3A. The image shows that the ink radius increases after one minute suggesting absorption of the solvent and ink particles into the fiber bulk. The image further shows that there is a decrease in the contact angle of the ink-to-fabric suggesting that the ink has begun to wet the substrate in the planar and through-plane directions. FIGS. 3D and 3E show images for the surface area at zero time and one minute after the ink droplet was deposited on an Evolon® nonwoven textile. FIG. 3F shows surface area data for ink drops a function of time based on images such as those shown in FIGS. 3B and 3C are quantitated as related to elapsed time following deposition of the drop. FIG. 3G shows surface area data for ink drops a function of time based on images such as those shown in FIGS. 3D and 3E are quantitated as a related to elapsed time following deposition of the drop.

FIGS. 4A-4B show representative scanning electron micrograph (SEM) images. FIG. 4A shows an SEM photomicrograph of a surface of an Evolon® nonwoven textile sample. The arrow in the upper right shows a non-split fiber; whereas the other arrows splitting of single fibers into multiple fibers at the polyester and polyamide interface, most likely via a process known as hydroentangling. FIG. 4B shows a cross-sectional image of the same textile sample. The arrows in this image highlight single fibers into multiple fibers at the polyester and polyamide interface. In each image there is a relative scale bar indicated for dimensional context.

FIGS. 5A-5C show representative data pertaining to the relationship of certain process variables, such as dispense velocity, fluid pressure, and viscosity, and printed line width for printing onto an Evolon® nonwoven textile. FIGS. 5A, 5B, and 5C show data obtained using a commercially obtained Ag/AgCl ink diluted to the indicated viscosity 0/10, 0.5/10 and 1/10. As described above for FIG. 2A, the viscosity refers to the amount of diluent used to dilute 10 gm of the native ink, respectively, i.e., dilution with 0 ml, 0.5 ml and 1.0 ml of diluent, 2-butoxyethyl acetate. FIG. 5A shows line width data for ink having viscosity 0/10 that was ejected at the indicated fluid pressures and dispense velocities. FIG. 5B shows line width data for using a disclosed Ag/AgCl ink with a viscosity of 0.5/10 that was ejected at the indicated fluid pressures and dispense velocities. FIG. 5C shows line width data for a disclosed Ag/AgCl ink with a viscosity of 1/10 that was ejected at the indicated fluid pressures and dispense velocities. The data show that as the viscosity decreases, the line width tends to increase. The data further show that as the dispense velocity increases, the line width tends to increase overall as the ink viscosity is decreased.

FIGS. 6A-6F show representative scanning electron micrographs (SEM) images pertaining to the penetration of inks having different viscosities under low and high fluid pressures. The images show a cross-sectional view. The study was carried out under the same conditions as those used to obtained the data in FIGS. 5A-5C with different ink viscosities as described therein. The images were obtained from samples in which the ink had been ejected at dispense velocity 70 mm/s. Each image has been labeled to distinguish a TPU film layer on the top, an Ag/AgCl ink layer underneath the TPU film interfacing to the top face of the nonwoven fabric, and a layer of ink which penetrates through the fiber bulk. The total ink height combined with the portion sitting on top of the textile and penetrating into the fabric is as indicated in the image. The horizontal or skewed horizontal line shows the position of the surface of the nonwoven fabric in order to easily distinguish the ink above the fabric surface and the ink that has penetrated the fiber bulk. FIG. 6A shows a SEM image obtained of a conductive ink printed using a disclosed Ag/AgCl ink with a viscosity of 0/10 ejected under a fluid pressure of 54 psi. FIG. 6B shows a SEM image obtained of a conductive ink printed using a disclosed Ag/AgCl ink with a viscosity of 0.5/10 ejected under a fluid pressure of 22 psi. FIG. 6C shows a SEM image obtained of a conductive ink printed using a disclosed Ag/AgCl ink with a viscosity of 1/10 ejected under a fluid pressure of 22 psi. FIG. 6D shows a SEM image obtained of a conductive ink printed using a disclosed Ag/AgCl ink with a viscosity of 0/10 ejected under a fluid pressure of 36 psi. FIG. 6E shows a SEM image obtained of a conductive ink printed using a disclosed Ag/AgCl ink with a viscosity of 0.5/10 ejected under a fluid pressure of 13 psi. FIG. 6F shows a SEM image obtained of a conductive ink printed using a disclosed Ag/AgCl ink with a viscosity of 1/10 ejected under a fluid pressure of 13 psi.

FIGS. 7A-7C show representative schematic views illustrating a model of migration of ink particles into the fiber bulk as ink viscosity varies. FIG. 7A shows a schematic view of a model of migration of ink particles into the fiber bulk for a higher viscosity ink (viscosity 0/10). FIG. 7B shows a schematic view of a model of migration of ink particles into the fiber bulk for a lower viscosity ink (viscosity 0.5/10). FIG. 7C shows a schematic view of a model of migration of ink particles into the fiber bulk for an even lower viscosity ink (viscosity 1/10). The schematic models show drop height, printed line width, and fiber bulk penetration as it relates to differing ink viscosity.

FIGS. 8A-8F show representative optical images of printed conductive inks corresponding to the SEM images shown in FIGS. 6A-6F. “LW” indicates the line width of the printed conductive ink shown in the image. A calibration bar is given in each image to provide dimensional context. FIG. 8A shows an optical image obtained of a conductive ink printed at an ink viscosity of 0/10 ejected under a fluid pressure of 54 psi. FIG. 8B shows an optical image obtained of a conductive ink printed using a disclosed Ag/AgCl ink with a viscosity of 0.5/10 ejected under a fluid pressure of 22 psi. FIG. 8C shows an optical image obtained of a conductive ink printed using a disclosed Ag/AgCl ink with a viscosity of 1/10 ejected under a fluid pressure of 22 psi. FIG. 8D shows an optical image obtained of a conductive ink printed at an ink viscosity of 0/10 ejected under a fluid pressure of 36 psi. FIG. 8E shows an optical image obtained of a conductive ink printed at an ink viscosity of 0.5/10 ejected under a fluid pressure of 13 psi. FIG. 8F shows an optical image obtained of a conductive ink printed at an ink viscosity of 1/10 ejected under a fluid pressure of 13 psi.

FIGS. 9A-9B show representative durability data for a representative disclosed direct write printed device. FIG. 9A shows data for normalized change in resistance versus number of cycles of electromechanical 90° bending tests on a direct write printed device. FIG. 9B shows data for normalized change in resistance versus wash cycle following AATCC standard 61-2a.

FIGS. 10A-10B show cross-section scanning electron micrograph (SEM) images for a representative wearable textile electronic device, i.e., a printed resistive heater. FIG. 10A shows a cross-sectional SEM image for printed resistive heater that was printed onto an Evolon® nonwoven textile at a fluid pressure of 42 psi and a speed of 70 mm/sec using a disclosed Ag/AgCl ink with a 0/10 viscosity. The thickness, as indicated in the figure, of the printed resistive heater is about 140 μm. FIG. 10B shows a cross-sectional SEM image for printed resistive heater that was printed onto an Evolon® nonwoven textile at a fluid pressure of 6 psi and a speed of 70 mm/sec using a disclosed Ag/AgCl ink with a 1/10 viscosity. The thickness, as indicated in the figure, of the printed resistive heater is about 45 μm. Each of the figures shows a relative scale bar for dimensional context.

FIGS. 11A-11E show representative images of the printed resistive heaters described above for FIGS. 10A-10B. FIG. 11A shows an optical image of the printed restive heater described for FIG. 10A, with a relative scale bar shown for dimensional context. FIG. 11B shows an optical image of the printed resistive heater described for FIG. 10B, with a relative scale bar shown for dimensional context. FIG. 11C shows an infrared thermal image of the printed resistive heater shown in FIG. 11A obtained after applying a voltage of 9 VDC. FIG. 11D shows an infrared thermal image of the printed resistive heater shown in FIG. 11B obtained after applying a voltage of 9 VDC. FIG. 11E shows an infrared thermal image of the representative disclosed printed resistive heater (as shown in FIG. 11A) placed on a shirt garment and worn by a subject.

FIG. 12 shows a representative plot of temperature generated for the representative printed resistive heaters described above. In the figure, the data shown for “Ink Viscosity 0/10” correspond to the printed resistive heater described in FIGS. 10A and 11A, whereas the data shown for “Ink Viscosity 1/10” correspond to the printed resistive heater described in FIGS. 10B and 11B. The voltage (VDC) applied to the indicated printed resistive heater is as indicated in the figure.

FIGS. 13A-13B show a further disclosed wearable textile electronic device, i.e., a meshed patch antenna, and reflection coefficient data. The inset shown in FIG. 13A shows printing of the meshed patch antenna, specifically, a print needle tip printing lines via a disclosed direct-write method using a disclosed Ag/AgCl ink with a 1/10 viscosity. FIG. 13B shows reflection coefficient data obtained at different frequencies using meshed patch antennas with the indicated printed conductive line widths obtained by printing at the indicated fluid pressures at a dispense velocity of 50 mm/sec. Data were modeled based upon the characteristics of the meshed patch antenna (dashed lines as indicated), showing a good correlation between modeled data and data obtained with the actual meshed patch antennas.

FIGS. 14A-14C show representative optical profilometry images characterizing the surface roughness and porosity of various textile substrates. FIG. 14A shows a representative optical profilometry image of the surface of a nonporous thermoplastic polyurethane laminate fabric material. FIG. 14B shows a representative optical profilometry image of the surface of an Evolon® nonwoven fabric material. FIG. 14C shows a representative optical profilometry image of the surface of a PET nonwoven fabric material.

FIGS. 14D-14I show representative images of droplets of a disclosed Ag/AgCl ink and the associated contact angles at zero time (i.e., immediately after deposition of the droplet) and five minutes later. The wetting behavior of the ink on each substrate, the contact angle of each substrate was also characterized immediately after deposition of an ink droplet and then after five minutes on the substrate surface (FIGS. 14D-14I). FIGS. 14A and 14B show a side view of a droplet deposited onto a thermoplastic polyurethane laminate at zero time and five minutes later, respectively, and the associated contact angles at the respective time. FIGS. 14C and 14D show a side view of a droplet deposited onto an Evolon® nonwoven fabric at zero time and five minutes later, respectively, and the associated contact angles at the respective time. FIGS. 14E and 14F show a side view of a droplet deposited onto a polyethylene terephthalate substrate at zero time and five minutes later, respectively, and the associated contact angles at the respective time.

FIGS. 15A-15E show data and images for representative disclosed printed resistive heaters. The printed resistive heaters were printed onto various substrates (polyethylene terephthalate (PET) nonwoven, indicated as “PET” in the figures; Evolon® nonwoven, indicated as “Evolon” in the figures; and a thermoplastic polyurethane laminate, indicated as “TPU” in the figures). The resistive heaters were printed using a disclosed a conductive Ag/AgCl ink with viscosity 10,000 cp (at 1 s⁻¹ shear rate) at a dispense velocity of 40 mm/s and a fluid pressure of 7 psi, followed by curing and heat pressing as described herein. A clear TPU film was used to package and encapsulate the printed conductive tracks at 150° C. for device protection and wearability. Under these conditions, the printed conductive lines on Evolon, PET, and TPU had line width of 938±5.2 μm, 875±41.14 μm, and 834.4±36.71 μm; and a printed conductive line thickness of 36.06 μm, 126.07 μm, and 86.65 μm. FIG. 15A shows temperature versus time of printed heaters on different substrates, as indicated, upon an applied voltage of 12 VDC. FIG. 15B shows sheet resistance data for the printed resistive heaters using the indicated substrate materials. FIG. 15C shows an infrared thermal image of a printed heater fabricated as described herein using a TPU substrate material. FIG. 15D shows an infrared thermal image of a printed heater fabricated as described herein using an Evolon® nonwoven fabric substrate material. FIG. 15E shows an infrared thermal image of a printed heater fabricated as described herein using a PET substrate material.

FIGS. 16A-16C show representative cross-sectional scanning electron micrograph (SEM) images for the printed resistive heaters discussed in FIGS. 15A-15E. FIG. 16A shows an image of a printed resistive heater on an Evolon® nonwoven fabric substrate with a TPU laminate over the printed resistive heater. The figure shows a relative scale bar indicated therein for dimensional context. Shown in the upper left corner of FIG. 16A is an inset image at greater magnification of the area indicated, with the thickness of printed resistive heater as indicated therein. FIG. 16B shows an image of a printed resistive heater on PET substrate with a TPU laminate over the printed resistive heater. The figure shows a relative scale bar indicated therein for dimensional context. Shown in the upper left corner of FIG. 16B is an inset image at greater magnification of the area indicated, with the thickness of printed resistive heater as indicated therein. FIG. 16C shows an image of a printed resistive heater on TPU substrate with a TPU laminate over the printed resistive heater. The figure shows a relative scale bar indicated therein for dimensional context. Shown in the upper left corner of FIG. 16C is an inset image at greater magnification of the area indicated, with the thickness of printed resistive heater as indicated therein.

FIG. 17 shows data for normalized change in resistance versus number of cycles of electromechanical bending tests (80% compression of the initial length) for the printed resistive heaters described in FIGS. 15A-15C and 16A-16C printed on the indicated substrates.

FIGS. 18A-18C show representative data for heating-cooling cycles of printed resistive heaters analyzed after 1000 cycles of bending (as described in FIG. 17) compared to the same sample prior to the 1000 cycles of bending. FIG. 18A shows comparative heating-cooling cycle for a printed resistive heater on a TPU substrate as described for FIGS. 15C and 16C. FIG. 18B shows comparative heating-cooling cycle for a printed resistive heater on an Evolon® nonwoven fabric substrate as described for FIGS. 15D and 16A. FIG. 18C shows comparative heating-cooling cycle for a printed resistive heater on a PET substrate as described for FIGS. 15E and 16B.

FIG. 19 shows representative normalized change in resistance versus number of washing/drying cycles for the printed resistive heaters described in FIGS. 15A-15C and 16A-16C printed on the indicated substrates.

FIGS. 20A-20B show representative infrared thermal images for a disclosed wearable textile electronic device, i.e., a printed resistive heater, obtained using a shirt garment worn by a subject. FIG. 20A shows an infrared thermal image of a printed resistive heater before application of voltage. FIG. 20B shows an infrared thermal image of a printed resistive heater obtained after a one minute application of 7 VDC (600 mA).

FIG. 21 shows a representative disclosed wearable textile electronic device, i.e., a printed resistive heater fabricated with a medical knee support which can be adapted for any joint or body part such as shoulders, back, neck, ankles, wrists, hip, legs, or body part/area that is in need of heat application for therapeutic or healing promotion. As shown in the figure, medical knee support comprises a disclosed printed resistive heater using a meandering pattern and further comprises a power source, such as rechargeable battery, wireless connectivity (e.g., Bluetooth, wifi, or other radiofrequency modes of wireless data transfer), and an application user interface that can connect with a smartphone or mobile device for ease of use and enhanced user experience to set time duration of heat or temperature of heat application.

FIGS. 22A-22C show a schematic representation for a disclosed nozzle set up for printing the vertical interconnect access (VIA) on a textile platform or substrate. As shown in the figure, there are VIAs (FIG. 22A) and interconnects (FIGS. 22B-22C) printed on both side of a suitable substrate, e.g., a needle-punched PET nonwoven laminated fabric.

FIGS. 23A-23C show representative images of printing a vertical interconnect access (VIA). FIG. 23A shows locations of needle punched sites on a needle-punched PET nonwoven laminated fabric that provide VIA points. The image further shows a needle directing a conductive ink, such as a disclosed Ag/AgCl ink, into a needle punched site to create a VIA. FIG. 23B shows a needle depositing a conductive ink between two connecting VIAs. FIG. 23C shows further deposition of conductive ink connecting multiple VIAs on the top surface of the needle-punched PET nonwoven laminated fabric.

FIGS. 24A-24D show schematic representations and images of a device with VIAs and connected interconnects on both sides of a nonwoven fabric. FIG. 24A shows a schematic representation of a disclosed wearable textile electronic device comprising VIAs and connected interconnects on both sides of a nonwoven fabric. The interconnect conductive lines had a conductive line thickness of about 856 μm and a conductive line width of about 1.5 to 2 mm. FIG. 24B shows an image of one surface of a disclosed wearable textile electronic device comprising VIAs and connected interconnects on both sides of a 1.2 mm needle-punched PET nonwoven fabric. FIG. 24C shows representative electrical resistance data (7.88Ω) obtained from end-to-end of the printed pattern for the device shown in FIG. 24B. FIG. 24D demonstrates that relative flexibility for the device shown in FIG. 24B.

FIGS. 25A-25C show representative cross-sectional scanning electron micrograph (SEM) images for printed VIAs on needle-punched PET nonwoven fabric of different thicknesses. Each image also shows an inset image at greater magnification showing penetration of the conductive Ag/AgCl ink into the fiber bulk, thereby creating a composite, electrically connected network vertically through the fiber bulk. A 1 mm relative scale bar is shown for each of the main images for dimensional context. FIG. 25A shows a cross-sectional SEM image for a VIA printed on a needle-punched PET nonwoven fabric with a nominal thickness of 1.2 mm. FIG. 25B shows a cross-sectional SEM image for a VIA printed on a needle-punched PET nonwoven fabric with a nominal thickness of 0.9 mm. FIG. 25C shows a cross-sectional SEM image for a VIA printed on a needle-punched PET nonwoven fabric with a nominal thickness of 0.35 mm.

FIG. 26 shows representative normalized change in resistance versus number of normalizednormalized change in resistance as a function of electromechanical bending cycles for a printed VIA on needle-punched PET nonwoven fabric of different thicknesses, as indicated in the figure.

FIG. 27 shows a schematic representation of a resistive heating device. The representative device has dimensions of about 4″×6″, with conductive lines with a line width of about 4-5 mm. The conductive lines can be fabricated from conductive ink using the disclosed direct-write printing methods. Alternatively, the conductive lines can be cut in the indicated pattern from a conductive fabric material. As shown in the figure, the conductive lines interface to a portable DC power source with capability to set time/temperature and wireless capability for mobile device connectivity as mentioned in [0030] (indicated by the component labeled “V”).

FIG. 28 shows a schematic representation of a resistive heating device. The representative device has dimension of about 4-5″×10-12 inches. Thus, this device encompasses about 40-60 in², compared to about 24 in² for the device shown in FIG. 27. In order to optimize performance for a device covering the larger area, as shown in FIG. 28, the device comprises a network of lower resistance conductive lines (i.e., the conductive lines shown as having a line width of about 5 mm), which are interconnected with higher resistance conductive lines (i.e., the conductive lines shown as having a line width of about 1-2 mm). The resistive heating device would generate resistive heat at the higher resistance interconnecting conductive lines, whereas the lower resistance conductive lines would generate almost no resistive heat by comparison due to the lower resistance of these lines. As shown in the figure, the conductive lines interface to a portable DC power source (indicated by the component labeled “V”).

FIG. 29 shows a schematic representation of a cross-sectional view of a wearable multilayer resistive heating textile package. The layer indicated as the breathable conductive textile/ink layer can comprise any resistive heating device disclosed herein, including those devices depicted in FIGS. 27 and 28. As shown in FIG. 29, the breathable conductive textile/ink layer is disposed in the multilayer structure comprises a first layer comprising a textile backing, a second layer comprising a thermally stable adhesive, a third layer comprising the breathable conductive textile/ink layer, a fourth layer comprising a permeable textile insulator, and a fifth layer comprising a comfort-textile layer. The multilayer resistive heating textile package can be further incorporated into a variety of articles, such as the wearable heating back wrap shown in FIGS. 30A-30C.

FIGS. 30A-30C each show a representative wearable heating back wrap. Highlighted in each of FIGS. 30A-30C are particular aspects of the given wearable heating back wrap. FIG. 30A shows one configuration of a wearable heating back wrap that can be secured about a torso via the back wrap closure, which can comprise a hook-and-loop closure, adhesive tape, or a strap and buckle design. FIG. 30B shows an alternative version of a wearable heating back wrap that is formed using a fabric comprising a stretchable fabric such as a spandex and that forms a continuous garment that can be secured about a torso due to the tension provide by the stretchable fabric. FIG. 30C shows a further alternative wearable heating back wrap configured with a series of batteries oriented approximately perpendicular to the spine, such that the wearable heating back wrap provides for bending mobility when engaged on a torso. The wearable heating back wrap shown in FIG. 30C further comprises a closure system comprising a hook-and-loop attachment.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

The present disclosure can be understood more readily by reference to the following detailed description of the disclosure and the Examples included therein.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, textile engineering, electrical engineering, and the mechanical arts.

### A. DEFINITIONS

As used in the specification and the appended claims, the singular forms “a,” “an” and “the” include plural referents unless the context clearly dictates otherwise. Thus, for example, reference to “a device,” “an ink,” or “a fabric” includes mixtures of two or more such devices, inks, or fabrics, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or can not occur, and that the description includes instances where said event or circumstance occurs and instances where it does not.

As used herein, “thermoplastic material” refers to a polymer that becomes pliable, moldable, and/or liquid above a threshold temperature and hard and/or solid when cold.

As used herein, “thermoset polymer” refers to a polymer based material made of monomers that polymerize (cure) when heated, subjected to a chemical reaction, or irradiated (e.g. exposure to UV light). Thermoset materials are typically liquid or malleable prior to curing.

As used herein, “conductive ink” refers to a material that can conduct electricity and can be molded into shapes and patterns and when set or dry results in an object or conduit.

As used herein, “about,” “approximately,” and the like, when used in connection with a numerical variable, generally refers to the value of the variable and to all values of the variable that are within the experimental error (e.g., within the 95% confidence interval for the mean) or within +−10% of the indicated value, whichever is greater.

As used herein, “electrical component” refers to any basic discrete device or physical entity in an electronic system, and includes without limitation to semiconductors, diodes, transistors, integrated circuits, optoelectronic devices (e.g. LEDs, OLEDS, opto-isolators, opto-couplers, photo-couplers, photodiodes, PJT, JFET, SCR, TRIAC, Zero-crossing, TRIAC, open collector, CMOS, IC, solid state relays, opto switch, opto interrupter, optical switchm optical interrupter, photo switch, photo interrupter), battery, fuel cell, power supply, photo voltaic device, thermoelectric generator, piezoelectric sensor or circuit, Van de Graff generator, resistors (e.g. power resistor, SIP, DIP resistor networks, Rheostat, potentiometer, trim pot, thermistor, humistor, photoresistor, memristor, varistor, voltage dependent resistor, MOV, resistance wire, Nichrome wire, heating element, capacitor (e.g. integrated capacitors, fixed capacitors, variable capacitors, special capacitors (e.g. power, safety, filter, light-emitting, motor, photoflash, and reservoir capacitors), capacitor networks/arrays), vricap diodes, inductors (e.g. coil, choke, variable inductor, saturable inductor, transformer, magnetic amplifier, ferrite impedances, beads solenoid, microphone), RC networks, LC networks, transducers, sensors (e.g. gas sensors, liquid sensor, chemical sensors, biomolecule sensors, and the like). LVDTs, rotary encoder, inclinometer, motion sensor, flow meter, strain gauge (e.g. piezoelectric or resistive), accelerometer, RTD, bolometer, thermal cutoff switch, thermocouple, thermopile, magnetometer, hygrometer, terminals, connectors, ultrasonic motors, piezoelectric devices, switch (e.g. SPST, SPDT, DPST, DPDT, NPNY, humidistat, thermostat, reed switch, relay, centrifugal switch, mercury switch, limit switch, micro switch, knife switch), fuse, and optical fiber and other waveguides. Other electrical components will be instantly appreciated by those of skill in the art. When coupled to or otherwise integrated with the flexible interconnects provided herein, the electrical component(s) can have any number of connection points to the flexible interconnect as practically implementable, which will be appreciated by those of ordinary skill in the art. It will also be immediately appreciated that the electrical component(s) can have one or more connection points to one or more than one (multiple) flexible interconnect(s).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

### B. METHODS FOR DIRECT-WRITE PRINTING OF CONDUCTIVE MATERIALS ON TEXTILES

In one aspect, the disclosure relates to methods for direct-write printing of conductive materials on a substrate material, such as a textiles. More specifically, in one aspect, the present disclosure relates to direct-write methods utilizing a drop on demand printhead dispensing at least one conductive ink. The textile can be composed of or include a fiber or filament comprising cotton, cellulose, a combination of cotton and cellulose, polyethylene terephthalate, polyamide, polyester, thermoplastic polyurethane, or any other class of polymeric fiber, or combinations thereof.

Disclosed are methods for forming a conductive material comprising applying a conductive ink with a printer to a substrate material; wherein the conductive ink comprises a conductive microparticle; wherein the conductive ink has a viscosity of about 10,0001 cps to about 30100,000+ cps when determined at a 1 s⁻¹ shear rate; wherein the printer comprises a drop on demand ink jet printhead comprising at least one nozzle; wherein the at least one nozzle tip is at a distance of about 0.1 mm to about 0.4 mm from the substrate material; wherein the conductive ink is dispensed from the at least one nozzle at a dispensing velocity of about 50 mm/s to about 200 mm/s; and wherein the conductive ink is dispensed from the at least one nozzle at a fluid pressure of about 1 psi to about 100 psi.

Direct-write printing is a promising method for printing continuous roll-to-roll large-area electronics directly onto flexible substrates with a one-step process, ruling out the need for complex and materials-intensive lithographic processes. However, direct-write printing conductive circuits on rough textile materials is severely limited due to textile's inherent high surface roughness and porosity. The presently disclosed methods provide a novel high-throughput strategy that meets the processing criteria for the textile electronics industry. In various aspects, the disclosed methods comprise a unique on-demand ink deposition process that surprisingly provides at least a ten-fold improvement over current state of the art direct-write on textiles.

Textile materials are uniquely positioned as substrates for flexible and printed electronic applications due to their absorption and wicking properties, breathability, flexibility, and wearability. Textile-based electronics include sensors, interconnects, heating elements, and antennas that range in application across the automotive, defense, medical, and consumer electronics industries (e.g., see references [1]-[3]). However, technological barriers to any textile electronic device include the need to satisfy high-throughput, low-cost, and high-performance needs of integrating the electronics that are commiserate with the stringent product requirements of the textile. Of particular interest for textile electronics is the ability to leverage the rapidly advancing printed electronics industry. Due to materials advancements (e.g., see references [4]-[7]) in the printed electronics industry, the intersection of printed electronics and textiles is a growing area of research and development within academia and industry. A barrier to entry of many printing techniques, such as screen printing, is the issue of scaling production up to industry standards and the ability to rapidly customize designs.

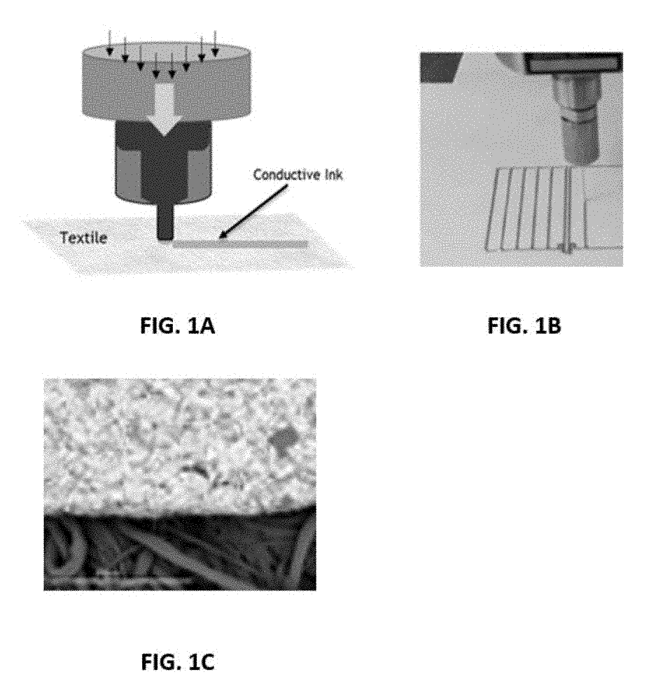

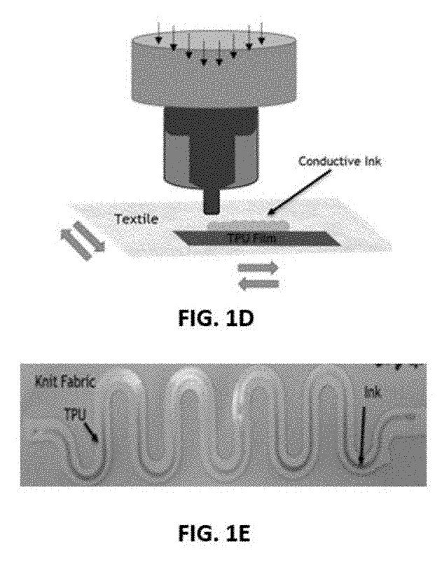

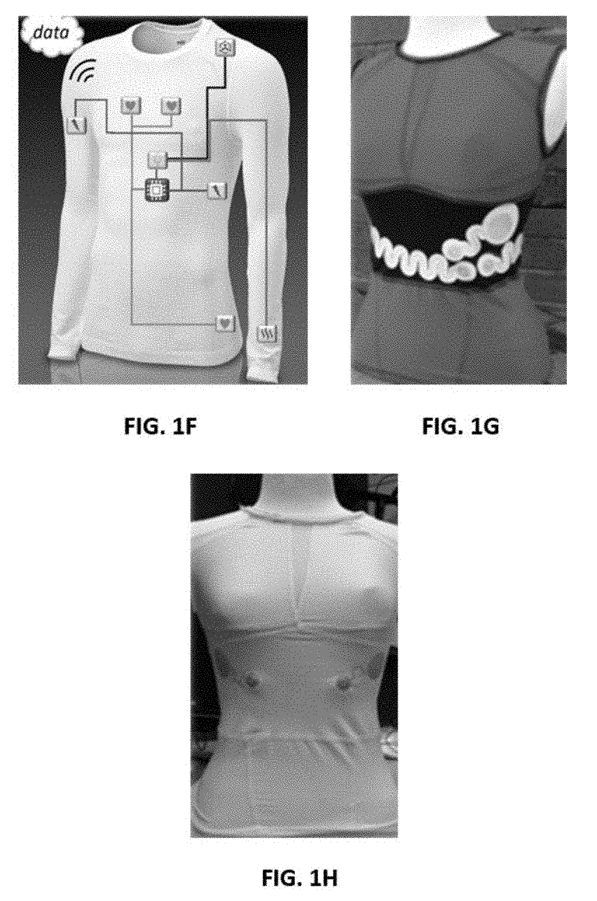

In contrast, the disclosed direct-write printing methods for use with textiles opens up the possibility for electronic textiles to be realized in a high-throughput manner using software driven designs and one-step material deposition technique without the need for making new screens, rollers, stamps, or masks. Accordingly, the disclosed direct-write printing methods can be used to automate the fabrication of textile electronics with multi-material deposition based on a software driven design process. In an exemplary aspect, the disclosed direct-write printing methods can be utilized to automate printing of wearable textile electronic devices such as wearable antennas (FIG. 1B; a representative photomicrograph of the nonwoven textile of FIG. 1B shown in FIG. 1C), interconnects (FIG. 1E), and multi-modal sensor systems for smart garments (FIG. 1F) in a single step process. In various aspects, the disclosed direct-write printing methods comprise one or more steps utilizing a drop-on-demand mode printer component (e.g., see FIG. 1A). In a further aspect, the disclosed direct-write printing methods comprise one or more steps comprising direct-write printing on a film such as thermoplastic polyurethane (TPU) as shown in FIG. 1D. In a still further aspect, the disclosed direct-write printing methods comprise one or more steps comprising direct-write printing on a film that is heat-laminated onto textile such as a polyester-spandex knitted textile.

In various aspects, the disclosed direct-write printing methods can be used to fabricate wearable textile electronic devices such as smart garments (see FIGS. 1F-1H). In a further aspect, a smart garment can be a garment configured to provide heart monitoring comprising a shirt and electronic components thereon and therein that have been printed using disclosed direct-write printing methods (FIG. 1F). In still further aspects, the disclosed direct-write printing methods can be used to fabricate meshed-patch antenna devices, e.g., disclosed direct-write printing methods in a direct-write drop-on-demand mode directly onto a textile such as non-woven textile (FIG. 1B). In yet further aspects, the disclosed direct-write printing methods can be used to fabricate heat-laminated interconnects such as heat-laminated interconnects configured in a meandering pattern (FIG. 1E). In other aspects, the disclosed direct-write printing methods can provide automated printing of textile electronics to fabricate garments comprising multi-modal sensing and/or energy harvesting components.

Conventional printing techniques in the electronics field include: screen-printing, inkjet printing, transfer printing, gravure printing, and direct-write printing [10]. Each of these printing techniques have specific advantages and disadvantages. Screen-printing is a technique that has been used in the textiles industry for hundreds of years and is well established for printing conductive patterns for circuitry on planar materials [11]-[12]. Screen-printing allows for a low-cost, accurate, and simple process printing of conductive patterns, however, it is susceptible to substantial ink waste, limited design flexibility, and limited printing area. Furthermore, device-to-device reliability can be limited due to deterioration of screen condition over time with repeated use. Transfer printing is another technique that has been used to print high-resolution conductive patterns. It utilizes a transfer device such as an ink stamp to print conductive patterns onto a textile substrate. Unfortunately, prior studies have not demonstrated the scalability of this printing method to meet the high-throughput requirements of the textiles industry [13].

Direct-write printing is segmented between droplet jetting and continuous filament writing. In droplet jetting (otherwise known as inkjet printing) the ink is deposited in a series of droplets onto the substrate to make a linear structure. Inkjet printing has advantages of printing on flexible substrates with precise control of line-width and film thickness. However, with textile substrates inkjet printing has proven to be a difficult process due to the need for multiple layers of ink printing (e.g., see [7], [14], and [15]). Moreover, inkjet printing requires low viscosity inks whose solvents and ink particles are absorbed by the textile substrate's fiber bulk, often prohibiting conductive percolation in the fibrous structure. In order to use inkjet printing reliably on textile substrates, currently available technologies require surface modifications be made to the textile to reduce the surface roughness and porosity to allow for improved adhesion of the ink particles on the textile surface (e.g., see [14] and [16]).

In continuous filament writing, the ink is deposited in a continuous filament structure onto the substrate. This technique allows fabrication using a computer-controlled pressure driven ink-suspension nozzle, permitting control of design and line dimensions on the substrate. This mechanism is very similar to extrusion-based 3D printing. In the direct-write process, the dispenser needle loaded with highly concentrated metallic ink is dispensed very close to the substrate in order to make continuous line patterns. However, on textiles it is extremely difficult to direct-write print due to the inherent high surface roughness of most textile materials which requires dispensing needle to be elevated enough from the textile substrate to avoid friction with the protruding fibers (e.g., see [17]-[18]). Previous work has demonstrated the process of direct-write on textiles, but was extremely limited in commercially applicability and scalability because it required up to five print passes to achieve suitable conductivity (0.0667 Ohms/cm) and print thickness (110 μm) (e.g. see [19]).

In contrast, the disclosed direct-write printing methods overcome the issues associated with commercial and manufacturing use with textiles noted above. In particular, the disclosed direct-write printing methods utilize an inkjet droplet jetting mode to increase throughput to connect individual droplets at high velocities unlike continuous mode printing. The disclosed methods provide optimized valve frequency for dispensing droplets. For example, in some aspects, the disclosed direct-write methods utilize a valve frequency of 77 Hz with a dispensing needle diameter of 0.25 mm, and a needle-to-substrate gap to 0.3 mm. Under the disclosed direct-write printing methods, deposition of highly viscous conductive inks at dispense velocities above 60 mm/s onto a textile substrate are achieved. Traditional inkjet printing utilizes droplet diameters on the scale of micrometers, which is smaller than most fiber diameters. In contrast, the disclosed direct-write process provides droplet sizes of about 1000 μm in diameter, which is greater than most fiber diameters thus allowing for a conductive percolation to be made at high-throughput.

In various aspects, the disclosed methods simplify the direct-write process technique to achieve commensurate line conductivity and thickness in a single printing stage. Disclosed herein are suitable printing process parameters (fluid pressure and dispense velocity) and the relationship of these parameters to conductive ink rheology. The disclosed direct-write system utilizes a droplet jetting technique and can potentially meet the high-throughput requirements of the textiles industry with optimum resolution of printed lines. The disclosed direct-write printing methods can utilize a textile material with suitable surface properties that are compatible with the disclosed screen-printable conductive inks.

In various aspects, the disclosed direct-write methods provide dispense velocities up to 80 mm/s. In contrast, currently available direct-write printing have only achieved up to 10 mm/s (e.g., [5], [17], and [20]). Moreover, the disclosed direct-write printing methods achieve a high dispense velocities (e.g., up to 80 mm/s) while at the same time providing a one print pass deposition process. Importantly, the disclosed direct-write printing methods are amendable to software driven printing methods suitable for rapid prototyping (e.g., software driven printing methods such as those described in [18]-[21]). The disclosed direct-write methods provide a high-throughput process for fabricating a multitude of textile electronic devices with a range of flexible substrates and conductive materials.

Without wishing to be bound by a particular theory, it is believed that solvent in the disclosed conductive ink can be absorbed by a textile allowing for the metal ink particles to percolate at the top layers of the textile substrate. Further, without wishing to be bound by a particular, it is believed that a micro-flake based ink can provide higher percolation compared to nanoparticle inks which would require higher metal loading to achieve similar conductivities. Thus, although it is possible to use nanoparticle based inks in the disclosed methods, for the foregoing reason, such inks may not be as efficient (i.e., may require printing thickness and width to achieve good conductivity), and would accordingly be more costly than a micro-flake based ink. The conductive ink can include a polymer binder. The polymer binder can be stretchable. The polymeric binder can be non-stretchable. The conductive ink can include a solvent suspensions system. The solvent suspension system can have a low vapor pressure for wettability to form ink-to-fiber composite structure.

In some aspects, the conductive inks can be cured. In some aspects curing can occur at a temperature of about 25° C. to about 150° C. for a period of about 1 minute to about 30 minutes. Curing can be conducted using a suitable technique. Suitable curing techniques include, but are not limited to, a radiation based curing process, a thermal based curing process, and combinations thereof. Other suitable curing techniques are described elsewhere herein and will be appreciated by those of ordinary skill in the art.

### C. WEARABLE TEXTILE ELECTRONIC DEVICES

In one aspect, the disclosure relates to articles such as wearable textile electronic devices comprising one or more components made using the disclosed direct-write methods. More specifically, in one aspect, the present disclosure relates an article comprising a component made by a disclosed method of direct-write printing of a conductive material onto a substrate material, such as a textile. In some aspects, the article comprises a component such as a printed resistive heating device, a printed sensor, a printed antenna, a vertical interconnect access, or combinations thereof, made by a disclosed method of direct-write printing of a conductive material onto a substrate material, such as a textile. In an aspect, the component is a textile based 2 GHz meshed-patch antenna. In a further aspect, the component is self-regulating wearable heating pad.

In various aspects, the article is a garment, an article of apparel, an article of footwear, an article of protective clothing, a helmet, a hat, a sock, a glove, a ballistic material, or an article of body armor. In a further aspect, the garment is a shirt, a pair of pants, an undergarment, or an article of outerwear.

In various aspects, the article is a medical device, a wound covering, a wound dressing, a medical mesh, or a medical fabric. In a further aspect, the medical device is an orthopedic support device selected from an arm brace, an elbow brace, back wrap or brace, or a knee brace.

In various aspects, the component is a printed circuit board or a connection to a printed circuit board. Printing flexible circuit boards are essential for integrating soft electronics such as sensors, actuators, energy harvesting devices, wireless devices onto a single platform. Unfortunately, currently available technologies are limited in being able to fabricate flexible printed circuit boards (PCBs) in which the electronics components (hard/soft) are mounted on both sides of the flexible substrate, such as a textile. The complexity and reliability of fabricating a vertical interconnect access which enables the integration of electronics on both sides of a substrate is presently the technology limitation for manufacturing flexible PCBs. Methods for fabricating VIAs have been described for thin substrates like paper in which holes are punched followed by metal deposition technique [42]. Alternatively, currently available techniques involve conducting multistep and complicated material deposition techniques [43]-[45]. Moreover, although these currently available methods are complicated and limited in their scalability, the reliability and robustness of such printed VIAs are not well described.

The disclosed high throughput direct-write printing process described herein can be used for fabricating VIA integrated flexible circuits on flexible nonwoven substrates with the thickness up to 1.2 mm.

### D. REFERENCES

The disclosure herein, including the Examples herein below, make reference to certain methods, procedures, compositions, and devices by citing the reference numbers herein below using the format of a reference number enclosed by “[ . . . ]” brackets.
1. Merritt, C. R.; Nagle, H. T.; Grant, E. Fabric-based active electrode design and fabrication for health monitoring clothing. IEEE Transactions on information technology in biomedicine 2009, 13, 274-280.
2. Suikkola, J.; Bjorninen, T.; Mosallaei, M.; Kankkunen, T.; Iso-Ketola, P.; Ukkonen, L.; Vanhala, J.; Mantysalo, M. Screen-Printing Fabrication and Characterization of Stretchable Electronics. Sci. Rep. 2016, 6, 25784.
3. Stoppa, M.; Chiolerio, A. Wearable electronics and smart textiles: a critical review. Sensors 2014, 14, 11957-11992.
4. Gao, Y.; Li, H.; Liu, J. Direct writing of flexible electronics through room temperature liquid metal ink. PLoS One 2012, 7, e45485.
5. Li, W.; Li, F.; Li, H.; Su, M.; Gao, M.; Li, Y.; Su, D.; Zhang, X.; Song, Y. Flexible Circuits and Soft Actuators by Printing Assembly of Graphene. ACS applied materials & interfaces 2016, 8, 12369-12376.
6. Matsuhisa, N.; Kaltenbrunner, M.; Yokota, T.; Jinno, H.; Kuribara, K.; Sekitani, T.; Someya, T. Printable elastic conductors with a high conductivity for electronic textile applications. Nature communications 2015, 6.
7. Gao, Y.; Shi, W.; Wang, W.; Leng, Y.; Zhao, Y. Inkjet printing patterns of highly conductive pristine graphene on flexible substrates. Ind Eng Chem Res 2014, 53, 16777-16784.
8. Khan, S.; Lorenzelli, L.; Dahiya, R. S. Technologies for printing sensors and electronics over large flexible substrates: a review. IEEE Sensors Journal 2015, 15, 3164-3185.
9. Shahariar, H.; Soewardiman, H.; Jur, J. S. In Fabrication and packaging of flexible and breathable patch antennas on textiles; SoutheastCon, 2017; IEEE: 2017; pp 1-5.
10. Parashkov, R.; Becker, E.; Riedl, T.; Johannes, H.; Kowalsky, W. Large area electronics using printing methods. Proc IEEE 2005, 93, 1321-1329.
11. Kazani, I.; Hertleer, C.; De Mey, G.; Schwarz, A.; Guxho, G.; Van Langenhove, L. Electrical conductive textiles obtained by screen printing. Fibres & Textiles in Eastern Europe 2012, 20, 57-63.
12. Karaguzel, B.; Merritt, C.; Kang, T.; Wilson, J.; Nagle, H.; Grant, E.; Pourdeyhimi, B. Utility of nonwovens in the production of integrated electrical circuits via printing conductive inks. Journal of the Textile Institute 2008, 99, 37-45.
13. Yoon, J.; Jeong, Y.; Kim, H.; Yoo, S.; Jung, H. S.; Kim, Y.; Hwang, Y.; Hyun, Y.; Hong, W.; Lee, B. H. Robust and stretchable indium gallium zinc oxide-based electronic textiles formed by cilia-assisted transfer printing. Nature communications 2016, 7.
14. Stempien, Z.; Rybicki, E.; Rybicki, T.; Lesnikowski, J. Inkjet-printing deposition of silver electro-conductive layers on textile substrates at low sintering temperature by using an aqueous silver ions-containing ink for textronic applications. Sensors Actuators B: Chem. 2016, 224, 714-725.
15. Chen, S.; Chiu, H.; Wang, P.; Liao, Y. Inkjet Printed Conductive Tracks for Printed Electronics. ECS Journal of Solid State Science and Technology 2015, 4, P3026-P3033.
16. Chauraya, A.; Whittow, W. G.; Vardaxoglou, J.; Li, Y.; Torah, R.; Yang, K.; Beeby, S.; Tudor, J. Inkjet printed dipole antennas on textiles for wearable communications. IET Microwaves, Antennas & Propagation 2013, 7, 760-767.
17. Ahmed, Z.; Torah, R.; Tudor, J. In Optimisation of a novel direct-write dispenser printer technique for improving printed smart fabric device performance; Design, Test, Integration and Packaging of MEMS/MOEMS (DTIP), 2015 Symposium on; IEEE: 2015; pp 1-5.
18. Bjorninen, T.; Virkki, J.; Sydanheimo, L.; Ukkonen, L. In Possibilities of 3D direct write dispensing for textile UHF RFID tag manufacturing; 2015 IEEE International Symposium on Antennas and Propagation & USNC/URSI National Radio Science Meeting; IEEE: 2015; pp 1316-1317.
19. Ahmed, Z.; Torah, R.; Yang, K.; Beeby, S.; Tudor, J. Investigation and improvement of the dispenser printing of electrical interconnections for smart fabric applications. Smart Mater. Struct. 2016, 25, 105021.
20. Lewis, J. A. Direct ink writing of 3D functional materials. Advanced Functional Materials 2006, 16, 2193-2204.
21. Li, Y.; Torah, R.; Beeby, S.; Tudor, J. Fully direct-write dispenser printed dipole antenna on woven polyester cotton fabric for wearable electronics applications. Electron. Lett. 2015, 51, 1306-1308.
22. Kranz, S.; Lewis, J. A. Multinozzle printheads for 3D printing of viscoelastic inks, 2013.
23. Ahn, B. Y.; Duoss, E. B.; Motala, M. J.; Guo, X.; Park, S. I.; Xiong, Y.; Yoon, J.; Nuzzo, R. G.; Rogers, J. A.; Lewis, J. A. Omnidirectional printing of flexible, stretchable, and spanning silver microelectrodes. Science 2009, 323, 1590-1593.
24. Wang, F.; Mao, P.; He, H. Dispensing of high concentration Ag nano-particles ink for ultra-low resistivity paper-based writing electronics. Sci. Rep. 2016, 6, 21398.
25. Yokus, M. A.; Foote, R.; Jur, J. S. Printed Stretchable Interconnects for Smart Garments: Design, Fabrication, and Characterization. IEEE Sensors Journal 2016, 16, 7967-7976.
26. Amendola, S.; Lodato, R.; Manzari, S.; Occhiuzzi, C.; Marrocco, G. RFID technology for IoT-based personal healthcare in smart spaces. IEEE Internet of Things Journal 2014, 1, 144-152.
27. HyungaCheong, W.; HyebBSong, J.; JoonaKim, J. Wearable, wireless gas sensors using highly stretchable and transparent structures of nanowires and graphene. Nanoscale 2016, 8, 10591-10597.
28. Misra, V., Bozkurt, A., Calhoun, B., Jackson, T., Jur, J. S., Lach, J., & Trolier-McKinstry, S. (2015). Flexible technologies for self-powered wearable health and environmental sensing. Proceedings of the IEEE, 103(4), 665-681.
29. Cho, G., Jeong, K., Paik, M. J., Kwun, Y., & Sung, M. (2011). Performance evaluation of textile-based electrodes and motion sensors for smart clothing. IEEE Sensors Journal, 11(12), 3183-3193.
30. Nateghi, M. R., & Shateri-Khalilabad, M. (2015). Silver nanowire-functionalized cotton fabric. Carbohydrate polymers, 117, 160-168.
31. Jin, L., Kim, K. J., Song, E. H., Ahn, Y. J., Jeong, Y. J., Oh, T. I., & Woo, E. J. (2016). Highly precise nanofiber web-based dry electrodes for vital signal monitoring. RSC Advances, 6(46), 40045-40057.
32. Perelaer, J., Smith, P. J., Mager, D., Soltman, D., Volkman, S. K., Subramanian, V., . . . & Schubert, U. S. (2010). Printed electronics: the challenges involved in printing devices, interconnects, and contacts based on inorganic materials. Journal of Materials Chemistry, 20(39), 8446-8453.
33. Chang, C., Tran, V. H., Wang, J., Fuh, Y. K., & Lin, L. (2010). Direct-write piezoelectric polymeric nanogenerator with high energy conversion efficiency. Nano letters, 10(2), 726-731.
34. Arnold, C. B., Serra, P., & Pique, A. (2007). Laser direct-write techniques for printing of complex materials. Mrs Bulletin, 32(1), 23-31.
35. Therriault, D., Shepherd, R. F., White, S. R., & Lewis, J. A. (2005). Fugitive inks for Direct-Write assembly of Three-Dimensional Microvascular Networks. Advanced Materials, 17(4), 395-399.
36. Yokus, M. A., & Jur, J. S. (2016). Fabric-based wearable dry electrodes for body surface biopotential recording. IEEE Transactions on Biomedical Engineering, 63(2), 423-430.
37. Lofhede, J., Seoane, F., & Thordstein, M. (2012). Textile electrodes for EEG recording—A pilot study. Sensors, 12(12), 16907-16919.
38. Merritt, C. R., Nagle, H. T., & Grant, E. (2009). Textile-based capacitive sensors for respiration monitoring. IEEE Sensors Journal, 9(1), 71-78.
393. Park, S., & Jayaraman, S. (2003). Smart textiles: Wearable electronic systems. MRS bulletin, 28(8), 585-591.
40. Cheng, Y., Zhang, H., Wang, R., Wang, X., Zhai, H., Wang, T., . . . & Sun, J. (2016). Highly stretchable and conductive copper nanowire based fibers with hierarchical structure for wearable heaters. ACS applied materials & interfaces, 8(48), 32925-32933.
41. Rahman, M. T., McCloy, J., Ramana, C. V., Panat, R. (2016). Structure, electrical characteristics, and high-temperature stability of aerosol jet printed silver nanoparticle films. Journal of Applied Physics, 120(7), 75305-7530511.
42. Byun, J.; Oh, E.; Lee, B.; Kim, S.; Lee, S.; Hong, Y. A Single Droplet-Printed Double-Side Universal Soft Electronic Platform for Highly Integrated Stretchable Hybrid Electronics. Advanced Functional Materials 2017, 27.
43. Zhang, Y.; Li, L.; Zhang, L.; Ge, S.; Yan, M.; Yu, J. In-situ synthesized polypyrrole-cellulose conductive networks for potential-tunable foldable power paper. Nano Energy 2017, 31, 174-182.
33. Jiang, D.; Sun, S.; Edwards, M.; Jeppson, K.; Wang, N.; Fu, Y.; Liu, J. A flexible and stackable 3D interconnect system using growth-engineered carbon nanotube scaffolds. Flexible and Printed Electronics 2017, 2, 025003.
45. Suarez, F.; Parekh, D. P.; Ladd, C.; Vashaee, D.; Dickey, M. D.; Ozturk, M. C. Flexible thermoelectric generator using bulk legs and liquid metal interconnects for wearable electronics. Appl. Energy 2017, 202, 736-745.

Before proceeding to the Examples, it is to be understood that this disclosure is not limited to particular aspects described, and as such may, of course, vary. Other systems, methods, features, and advantages of foam compositions and components thereof will be or become apparent to one with skill in the art upon examination of the following drawings and detailed description. It is intended that all such additional systems, methods, features, and advantages be included within this description, be within the scope of the present disclosure, and be protected by the accompanying claims. It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Direct-Write Printing Methods

Textile Substrate.

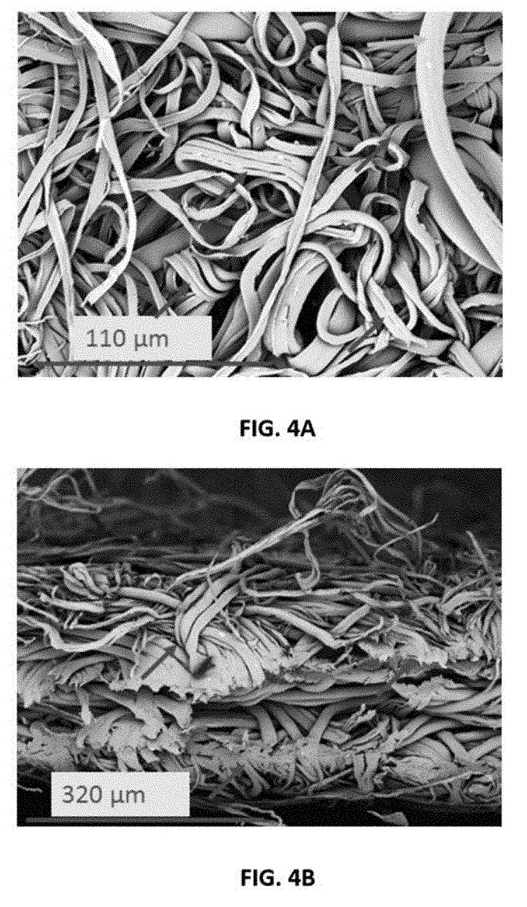

Evolon® nonwoven fabric having a basis weight of 115 g/m², manufactured by Freudenberg Performance Materials (Weinheim, Germany), was used as the primary textile substrate for printing conductive lines in the studies described in this section. The average surface roughness measured as about 18 μm and the surface area of about 8 mm² in 3.9 mm² fabric area. The choice of this material was due to its high absorbency. Evolon® nonwoven is manufactured by extrusion of two polymeric fibers (30 wt % of polyamide and 70 wt % polyester). The extruded bi-component fibers are bonded by high pressure water-jet that splits both polymeric fibers at the interface as shown in FIGS. 4A and 4B. The result fabric has a smooth surface with high surface area and a wedge-like microstructure. The engineered manufacturing process imparts high absorbency due to the capillary force of the split micro-fibers.

Textile Substrate:

Knitted Textile. In various embodiments the textile substrate can also be a knitted textile which has various ranges of surface roughness, porosity, fiber content and polymer type, and mechanical properties. A common knitted textile studied is a polyester-spandex knit textile with (88% polyester, 12% spandex). The direct-write process described herein can be utilized to print directly onto the given textile. An ink-to-fiber composite can be fabricated by altering the ink viscosity and ink wettability to penetrate into the fiber bundles to tune the resulting electromechanical properties of devices such as interconnects or sensors.

Printing Apparatus.

The studies described in this section used for printing conductive tracks on textiles a modified Nordson Asymtek conformal coating system (Model C-341) using a drop-on-demand mode. The three dimensional movement of nozzle on the Nordson Asymtek conformal coating system is controlled by a robotic hand that has a translation speed up to 508 mm/sec in X-Y axis and 203 mm/s in the Z axis. In order to obtain a high-throughput dispense velocities, the needle-to-substrate gap Asymtek conformal coating system was adjusted to a needle-to-substrate gap of 0.3-1.5 mm to allow the needle to operate at the desired throughput without coming into contact with protruding fibers of the textile substrate.

Conductive Ink.

The studies described in this section used a micro-flake based Ag/AgCl ink (Product No. 124-36; Creative Materials Incorporated, Ayer, Massachussetts, USA). The Ag/AgCl ink had Ag:AgCl ratio of 66:34, and a nominal viscosity of 17,000-23,00 cps. The ink was diluted using 2-butoxyethyl acetate as a diluent (Product No. 102-03; Creative Materials). In the described studies, three different viscosities of Ag/AgCl ink were utilized. The first viscosity sample, designated as viscosity 0/10, was undiluted Ag/AgCl ink as obtained from the manufacturer. The second and third viscosity samples of the conductive ink used in these studies were prepared by diluting 10 gm of Ag/AgCl ink with 0.5 mL and 1 mL of diluent and designated herein as viscosity 0.5/10 and viscosity 1/10, respectively. The ink used was a micro-flake based ink. Without wishing to be bound by a particular theory, it is believed that solvent in this conductive ink can be absorbed by the Evolon® textile due to the high-absorbency of this fabric, thereby allowing metal ink particles to percolate at the top layers of the textile substrate. Further, without wishing to be bound by a particular, it is believed that a micro-flake based ink can provide higher percolation compared to nanoparticle inks which would require higher metal loading to achieve similar conductivities.

Process Testing.

The textile fabric, a nonwoven Evolon® material as described above, was cut into samples with an area of 25 cm². Ink samples were loaded into the syringe barrels of the Nordson Asymtek conformal coating system and calibrated by printing four lines of 5 cm in length. Process studies were carried out to assess the ink-to-substrate relationship with respect to the dispensing velocity (mm/s) and ink-fluid pressure (psi). The Asymtek conformal coating system used in these studies utilized a 22 gauge (0.5 mm diameter) needle maintained at a 0.3-1.5 mm off of the surface of the fabric to avoid any friction with protruding fibers on the textile surface. Table 1 shows the process variables that were assessed.

TABLE 1
Process variables for the direct-write process on textile.

| Sample | Ink Viscosity (Pa · s | Fluid Pressure | Dispensing |
|---|---|---|---|
| designation | at 1 s⁻¹ shear rate) | (psi) | Speed (mm/s) |
| Viscosity 0/10 | 32.91; | 36, 42, 48, 54 | 60, 70, 80 |
| Viscosity 0.5/10 | 22.15; | 13, 16, 19, 22 | 60, 70, 80 |
| Viscosity 1/10 | 12.16; | 13, 16, 19, 22 | 60, 70, 80 |

After conductive lines were printed, the samples were heat-pressed for 5 minutes at 150° C. using a desktop heat-press and then allowed to cool for 2 minutes. It was determined that as printed, the conductive lines were prone to cracking, which rendered that conductive pattern inoperable. The problem of conductive line cracking was resolved by encapsulating the conductive lines with thermoplastic polyurethane (TPU) film (Product No. TL 3916; Bemis Company, Inc. Neenah, Wis.) having a nominal thickness of 150 μm. Encapsulation of the printed conductive lines was carried out by heat pressing the TPU film encapsulate on the ink patterns for 2 min at 125° C. with 2 min of cooling. The TPU encapsulate resolved the cracking behavior observed with unencapsulated conductive lines, but did not alter electrical resistance in the conductive lines.

SEM Characterization Method.

Textile samples with conductive lines were characterized by imaging with a Verios Scanning Electron Microscope (Analytical Instrumentation Center (AIF), North Carolina State University, Raleigh, N.C.). Due to the high conductivity of the fabric samples with conductive lines, SEM images were obtained using a voltage of 10 kV and a current at 1.6 nA.

Sheet Resistance Measurement.

Sheet resistance values were measured using experimentally obtained bulk resistance values for the conductive ink after heat pressed to dry at 120° C. for 2 min. The equation given immediately below (Equation 1) was used to calculate the sheet resistance, where R, Rₛ, p, t, L and W is the bulk resistance, sheet resistance, resistivity, thickness of the ink, length and width of the printed line, respectively.

$$R = \frac{p}{t} \frac{L}{W} = {R}_{s} \frac{L}{W} \qquad ( Eq . 1 )$$

Line Width Characterization.

Line widths were characterized using an optical microscope and ImageJ visual analysis software (an open source image processing program). A sampling average of 10 lines were measured on ImageJ to compute an average and standard deviation value for the given sample's line widths.

Direct-Write Printing.

Key to the understanding of the direct-write process and the available modes of printing is the understanding of the material properties, flow properties, and the process parameters for the extrusion of thixotropic, non-Newtonian fluids (such as conductive inks) from a nozzle orifice [22]. Highly concentrated colloidal suspensions of silver micro-flake particle ink show shear-thinning viscoelastic properties and yield-stress behavior. The thixotropic material flows well under high shear-stress and behaves like a solid below the yield point of the shear-stress. This behavior is best explained by the equations below (Equations 2 and 3):

η=K\dot{γ}ⁿ⁻¹ (Eq. 2)

τ=τ_{y}+K\dot{γ}ⁿ⁻¹ (Eq. 3)

where η is the viscosity, z is the yield stress, and \dot{γ} is the shear-rate. Equation 2 above shows that the viscosity is a function of the shear-rate, which decreases with an increase in the shear-rate. Equation 3 shows the stress-strain relationship with a non-zero yield-stress term. This indicates that when the material is at rest, it has an intrinsic yield-stress. Given that the conductive ink material is a colloidal suspension of silver micro-flake particles in a polymer binder and solvent, we characterized the material's ink rheology to understand the viscosity and shear-thinning viscoelastic behavior.

Ink Rheology.

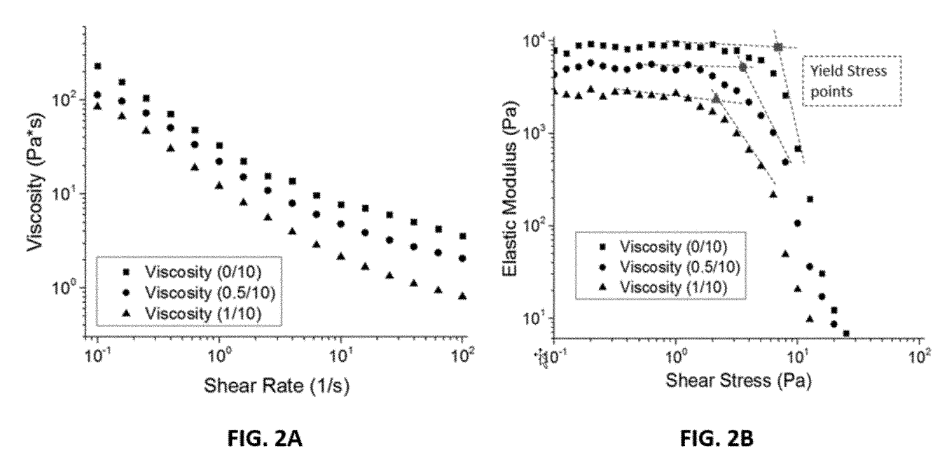

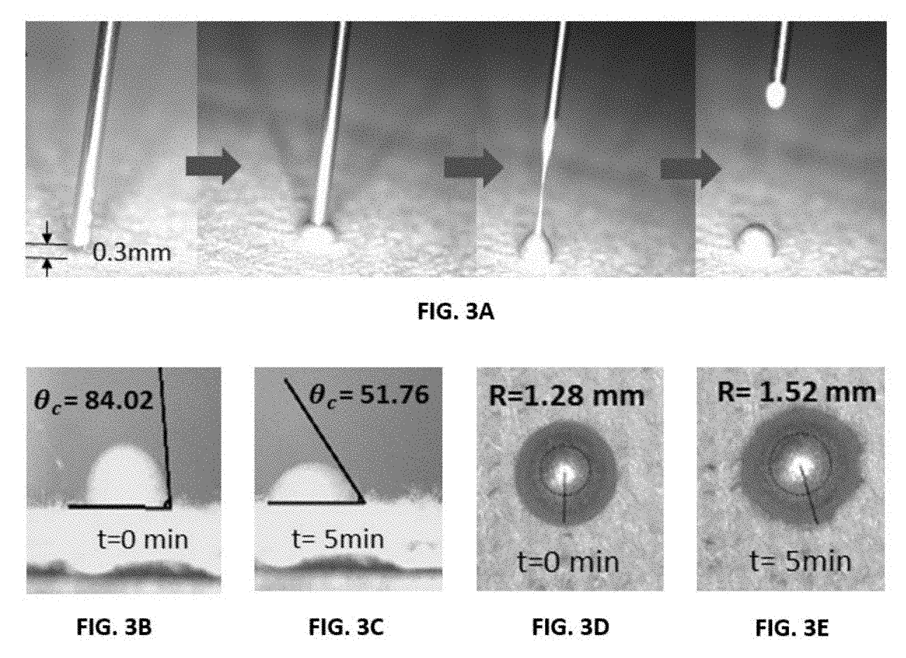

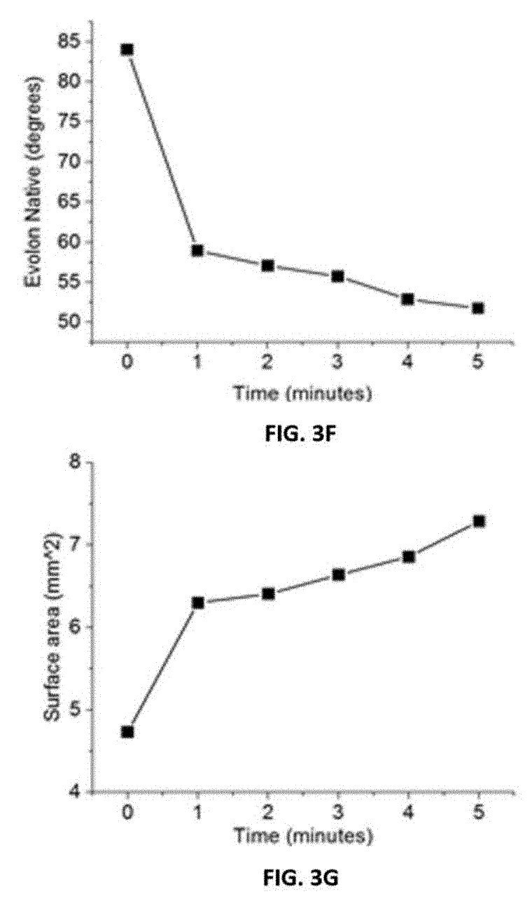

As the viscosity of the Ag/AgCl ink is decreased via dilution with the diluent, it may be predicted the viscosity would decrease and the shear stress needed to print would also decrease. As such, the understanding of the rheological behavior of these inks is critical for their use in printing applications such as direct-write printing. The flow behavior of the Ag/AgCl ink is shown in FIG. 2A, which demonstrates the shear thinning behavior of the ink. The shear thinning behavior means the ink will flow faster through the nozzle at a high shear rate. As the inks are diluted, the viscosity with corresponding shear rate decreases. FIG. 2B shows that all three ink viscosities tested (0/10, 0.5/10, and 1/10, as described herein above) have almost constant elastic modulus at low shear stress which decreases after crossing a critical shear stress, which is the yield stress as described in equation (3) above. For colloidal suspensions, the yield stress defines the point at which the network of particulates break and begin to flow. As such, the analyzed viscoelastic ink samples are able to flow through the nozzle when the shear stress (or back pressure) is beyond the yield stress value. FIG. 2B indicates three different ranges of yield stresses for the different viscosities of inks. The Ag/AgCl ink used herein as obtained from the manufacturer is highly viscous (i.e., the viscosity 0/10 sample) and requires a high yield stress to decrease the elastic modulus to the range where it can be printed (about 10 Pa). The yield stress for the lower viscosity inks is substantially lower. Below the yield stress point, the elastic modulus of the ink does not change with shearing and acts like a solid material. A minimum yield stress must be exerted to allow for the non-newtonian thixotropic conductive inks to flow and thus allow for printability. Further dilution of the ink breaks the suspended colloidal formation and results in poor resolution. Due to yield stress phenomena, the ink droplets tend to retain their shape after the applied shear stress is eliminated upon coming out of the nozzle. The inks studied herein exhibit this yield stress behavior, and thus this behavior is expected in every case (FIG. 3A).

Ink-to-Textile Interaction.

As the ink is dispensed from the print nozzle, the ink resembles a bead shaped droplet, a behavior that arises from the yield stress property of the ink. The ink maintains a constant positive value of elastic modulus even after the shear stress is reduced right after coming out of the nozzle. This helps to maintain the shape of the ink droplet. It was observed that the drop-on-demand (DoD) actuation mode was able to reliably maintain continuous printed lines at high velocities with considerably high resolution (0.6 mm-1.0 mm across a given length) on textile substrates. The nonwoven textile used in this study, Evolon®, has a very smooth surface (low surface roughness about 15 μm) with high surface area which imparts very strong wicking properties to the fabric as shown in FIGS. 3B-3E. The interaction of an ink droplet with the fabric substrate is important to understand the line resolution of the printed structures. An ink droplet on the Evolon® substrate spreads initially in the in-plane and through-plane direction of the fabric while the solvent of the ink is wicked by the fibrous structure of the fabric. However, the ink droplet stabilizes its shape which helps to conform good resolution of printed lines. FIG. 3A shows the stabilization of the ink as it is deposited onto the fabric.

FIGS. 3B and 3C shows the contact angle experiments done on the Evolon® nonwoven textile immediately after deposition of an ink drop and then after one minute on the textile surface, respectively. The images show a decrease in the contact angle of the ink-to-fabric suggesting that the ink has begun to wet the substrate in the planar and through-plane directions. FIGS. 3D and 3E shows a top-view of an ink drop deposited on the textile substrate immediately after deposition and then after one minute on the textile surface, respectively. It was observed that the ink radius increases after 1 minute suggesting absorption of the solvent and ink particles into the fiber bulk. FIG. 3F shows data for the contact angle of the ink droplet to the Evolon® surface with respect to time. The data show that the greatest decrease in the contact angle occurred in the first minute. FIG. 3G The left figure shows the surface area of the ink droplet on Evolon® with respect to time. It can be observed that greatest increase in surface area occurred in the first minute.

Optimization of Direct-Write Parameters for Electronic Patterns.

In the studies in this section, the stress behavior of the conductive fluid was characterized with respect to viscosity in order to understand optimal fluid pressure ranges and then an individual fluid droplet's interaction with the textile was understood through contact angle observations. The data obtained was utilized to determine the optimal gap between needle-to-textile surface and the droplet ejection frequency. In the studies herein, the Ag/AgCl conductive ink deposited or printed onto the nonwoven Evolon® textile had an optimal needle-to-textile gap of a 0.3 mm and an optimal droplet ejection frequency of 77 Hz droplet ejection frequency for printing continuously connected droplets which yielded continuous lines. These findings allowed for the substantial increase in throughput compared to other reported studies (see references [5], [17], [19], [22], and [23]).

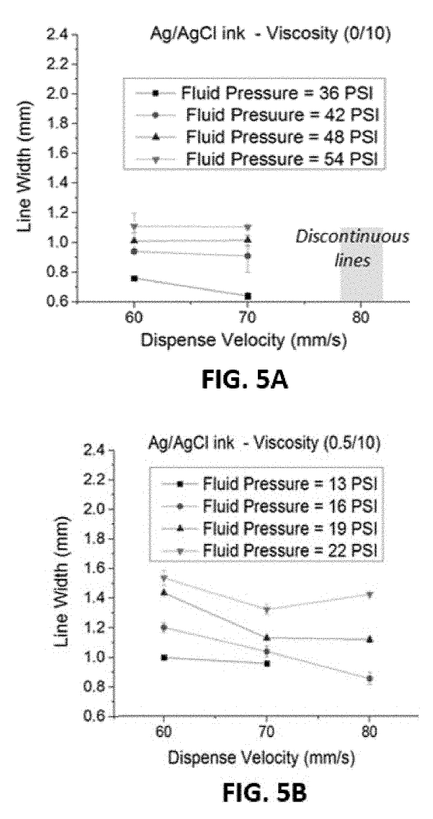

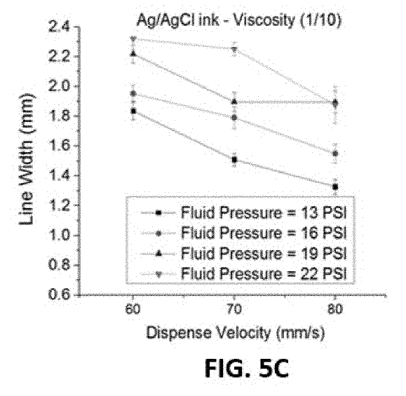

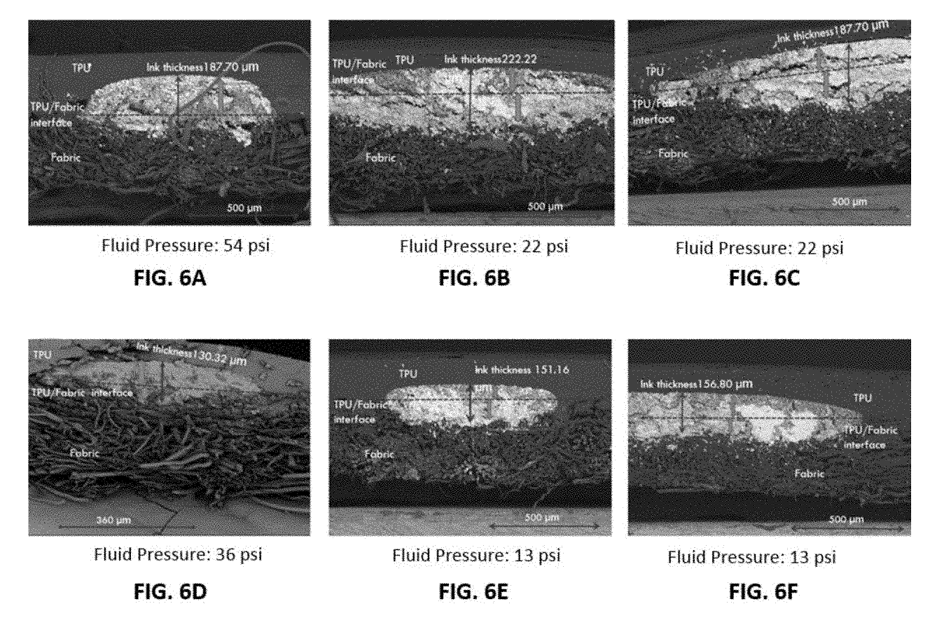

The resolution and uniformity of the printed lines by direct-write printing was analyzed in order to determine whether the disclosed methods could be used for printing electronics. Process parameters were analyzed with respect to the Evolon® surface properties and the rheological properties of the Ag/AgCl ink. FIGS. 5A-5C show the relationship between process variables of dispense velocity, fluid pressure, and viscosity with respected to the printed line widths. The line width increases as the ink viscosity decreases. Printed lines with viscosity 0/10 had to be printed at a high fluid pressure range (36-54 psi) and accordingly this ink viscosity produced the lowest line width. It was observed in this experiment, with the aid of SEM images and visual inspection that inks with lower solvent concentration showed negligible flow in the in-plane direction of the textile surface. The line width decreased as dispense velocity increased for all ink viscosities. The lowest observed line width was about 0.6 mm and was produced by ink viscosity 0/10 at a fluid pressure of 36 psi and at a dispense velocity of 70 mm/s. Discontinuous lines were observed at dispense velocity ≥80 mm/s for ink viscosity 0/10. This same phenomenon was observed again with ink viscosity 0.5/10 at dispense velocity 80 mm/s and at a fluid pressure of 13 psi. Line width was observed to be higher for ink viscosity 1/10 due to the ink flowing in the in-plane direction by the capillary wicking behavior of the Evolon®. It can also be observed that as the dispense velocity increases, the line width decreases when the ink is diluted. The results in FIGS. 5A-5C are further confirmed in the SEM images shown in FIGS. 6A-6F.

Ink Penetration and Spreading on a Textile Substrate.

The resolution and uniformity of the printed lines is not only defined by length and width, but also by the vertical dimension. Ink penetration into the textile is an important phenomenon that was observed with the direct-write process studies herein. It was observed that the ink penetrates below the textile's surface, creating wire-like channels in the textile, as well as above the textile. The penetration of the ink varies with different ink viscosities, fluid pressures, and dispense velocities.

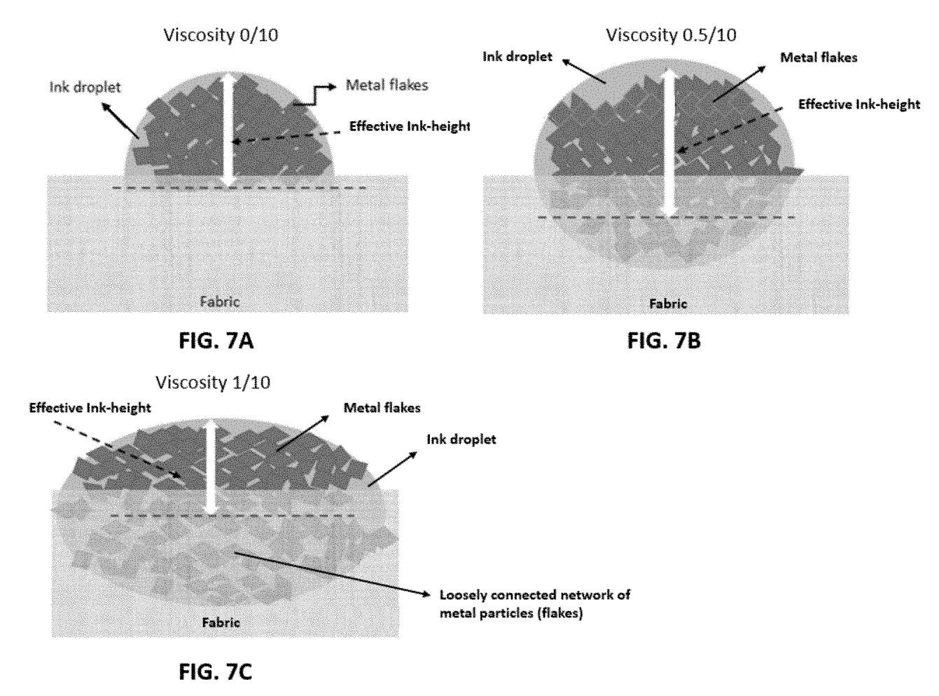

The scanning electron microscopy (SEM) of the cross-section of printed lines in FIGS. 6A-6F show the variation of ink penetration and the morphology of the ink from the printed lines produced at dispense velocity 70 mm/s. FIGS. 6A-6F further show the cross-section view of the printed lines with ink viscosity 0/10, 0.5/10 and 1/10, respectively. Each SEM image in FIGS. 6A-6F is labeled to distinguish the encapsulating TPU film layer on the top, an Ag/AgCl ink layer beneath the encapsulating TPU film and in interface with the top surface of the fabric, and a layer of ink that penetrates through the fiber bulk. The SEM images show only the cross-section of the lines printed with highest and lowest fluid pressure to show better contrast of the images. As the fluid pressure increases from 36 psi to 54 psi (compare FIGS. 6A and 6D), the ink thickness increases for ink viscosity 0/10. Due to having high ink viscosity, the penetration is not as high as that of the lower viscosity ink samples because the higher solid loading of the ink allows it to stay on top surface of the fabric surface and retain the wire-like structure as observed. FIGS. 6B and 6E show the ink penetration and ink thickness of conductive lines printed using ink viscosity 0.5/10. It can be observed that this ink spreads much more than the undiluted ink. There are also observed an increase in cracks in the cross-sectional interface of the ink and fabric composite structure as the fluid pressure is increased from 13 psi to 22 psi. FIGS. 6C and 6F show the penetration of least viscous 1/10 ink sample. It can be observed that the highest spreading of the ink occurred in this sample and this is further confirmed in line width data in shown FIGS. 5A-5C, and shown schematically in FIGS. 7A-7C. As the fluid pressure is increased from 13 psi to 22 psi the ink spreads in a non-uniform structure through the fiber bulk as evidenced by image c which results in many cracks. Without wishing to be bound by a particular theory, these data suggest that when ink with lower viscosities undergo a high fluid pressure, there will be more non-uniform spreading into the fiber bulk, resulting in higher ink penetration and resolution.

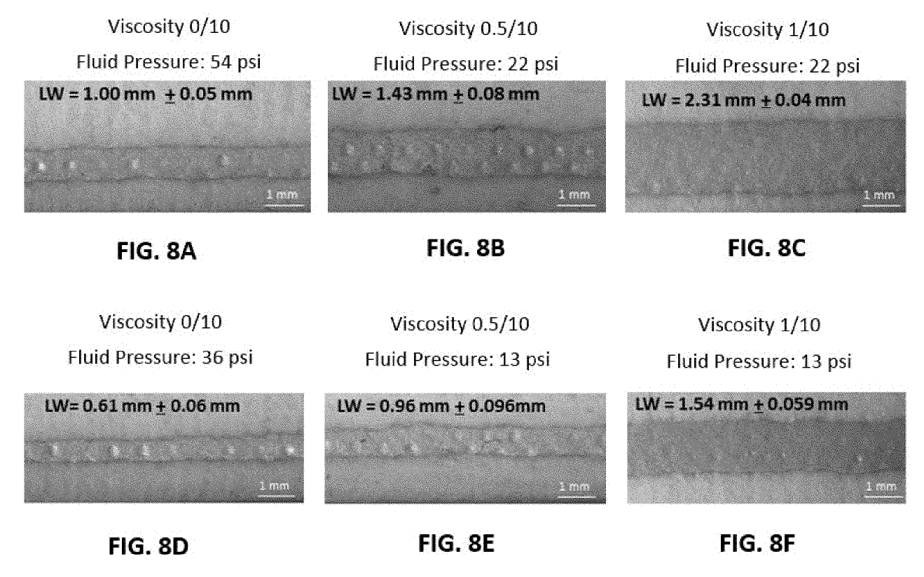

FIGS. 7A-7C show a model of the particle-flake loaded ink penetration or migration phenomena with respect to different ink viscosities. For ink viscosity 0/10, the ink does not penetrate as much into the fabric compared to lower viscosity ink tested. This ink also does not spread as much in the in-plane direction of the fabric. As a result, the printed line with ink viscosity 0/10 has lowest line width (see FIGS. 8A-8F). The effective ink height is higher for printed lines with ink viscosity 0.5/10 because the lower viscosity of the ink allows the ink to flow out more. Furthermore, ink viscosity 0.5/10 allows the ink to penetrate into the fabric retaining the continuous ink structure which results in having the lowest sheet resistance for the printed lines (see Table 2). Printed lines with ink viscosity 1/10 have the highest ink penetration into the fiber bulk than that of ink viscosity 0/10 as this has the highest flow of ink deposition into the fabric from our observations. However, the effective ink height is lower than what is visible in the SEM images because the diluted ink cannot retain its structure inside the fiber bulk. This phenomenon can be observed as visible cracks in the ink area (compare FIGS. 6A-6F, viscosity 1/10, 22 psi) which can hinder the electron flow and decrease the effective ink height of the printed lines. The data show that the highest sheet resistance was obtained from a conductive line printed using ink viscosity 1/10 (see Table 2).

Optical images are shown in FIGS. 8A-8F of printed lines corresponding to the SEM images shown in FIGS. 6A-6F.

Table 2 reviews the variation of sheet resistance and ink height of the printed lines. It is known that sheet resistance is primarily a function of the length, width, and height of the conductive film. The effective height of the conductive ink contributes to the electron flow through the printed lines. The height is calculated by using the measured sheet resistance of printed lines and the ink resistivity value as reported by the manufacturer (0.0002 Ω-cm). Thus, the higher the ink height of the printed line with equal planar surface area, the lower the electrical resistance. The total ink height comprises with the portion sitting on top of the textile and penetrating into the fabric is shown in FIGS. 6A-6F.

As shown herein, ink penetration and spreading in the fiber bulk are affected by the change of ink viscosities. For all of the ink viscosities tested, the height of the ink generally increases as the fluid pressure increases. The ink height of the printed line with ink viscosity 0/10 is mostly observed on the top of the fabric. As such, sheet resistance is higher for lines printed with ink viscosity 1/10 which had a higher surface area and lower effective height of the ink. As expected, the effective height is not the same as the observed total height from the SEM images. It is observed that the effective ink height is higher than the total ink height because the calculated effective ink height is based on the observed width from the optical images. However, the width can be higher due to the ink penetration in the in-plane and through-plane directions. Conversely, the effective height is only significantly lower for the sample printed with ink viscosity 1/10 at a fluid pressure 22 psi. The analysis suggests a possible disconnect between the observed and effective height values (see FIG. 6C). Thus, it possible that a portion of the printed line that does not contribute to charge transport which is also explained in FIG. 7C, thus producing a higher sheet resistance.

TABLE 2
Effect of process parameters on printing properties.

| | | | | Effective |
|---|---|---|---|---|
| | Fluid | Sheet | Total Ink Height | Ink Height |
| Ink | Pressure | Resistance | (from SEM image) | (calculation) |
| Viscosity | (psi) | (mΩ/sq) | μm | μm |
| Viscosity | 36 | 10.58 ± 0.07 | 130.32 | 145.23 |
| 0/10 | 54 | 9.91 ± 0.70 | 187.70 | 195.50 |
| Viscosity | 13 | 9.51 ± .40 | 151.16 | 188.13 |
| 0.5/10 | 22 | 9.56 ± 0.50 | 222.22 | 209.17 |
| Viscosity | 13 | 11.92 ± 0.70 | 156.80 | 167.70 |
| (1.0/10) | 22 | 15.49 ± 0.40 | 187.70 | 129.11 |

Durability of Printed Conductive Inks.

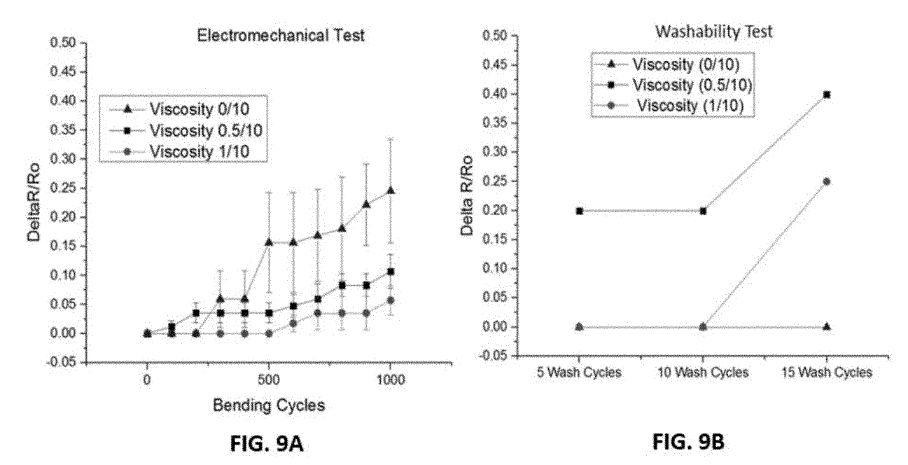

Cyclical electromechanical bend testing was conducted at a 90° folding angle. For comparison, similar work to show electromechanical behavior over 1000 bending cycles has been performed with direct-write printing of highly concentrated Ag nanoparticles on paper, showing a 20% increase in resistivity after testing [24]. As shown in FIG. 9A, viscosity 1/10 has the least change in resistance over 1000 bending cycles while viscosity 0/10 has the highest change in resistance. The results obtained with the ink viscosity 0/10 were comparable data reported for write printing of highly concentrated Ag nanoparticles on paper [24]. The robustness of the printed lines with viscosity 1/10 is due to the spreading and penetration of the conductive ink into the textile structure to make a composite structure that gives mechanical flexibility as opposed to viscosity 0/10 where cracking can occur due to the conductive ink sitting on top of the textile surface.

As for washability, accelerated wash cycle testing was carried out in according with AATCC standard 61-2a and the data are shown in FIG. 9B. The data show that viscosity 0/10 exhibited the best washability performance. Without wishing to be bound by a particular, it is possible that this superior performance was due to higher viscosity conductive ink having a more well-defined structure with less spreading and penetration into the textile as shown in FIGS. 6A-6F. As a result, less water and detergent was able to penetrate and loosen the conductive ink particles. The sample with the least change in electric resistance was viscosity 0/10 and the sample with the most was viscosity 0.5/10.

2. Wearable Textile Electronic Devices

Printed Resistive Heater.

Printed and flexible heating elements are of great interest for wearable technology applications such as in garments or in automotive heating. The disclosed direct-write printing process was used to study the potential of fabricating printed resistive heating devices for wearable applications. The printed resistive heaters tested herein have an area of 105.5 cm² with total track length of 166.68 cm of a meandering printed line. For this demonstration, the process variables were tuned to achieve a printed line with approximately the same width and length as discussed herein above, but with different ink heights as shown in Table 3. To maintain similar X-Y resolution of printed heaters, the Test Heater No. 1 was printed using ink viscosity 0/10 at 42 psi and 70 mm/s. Test Heater No. 2 was printed using ink viscosity 1/10 at 6 psi and 70 mm/s. As discussed herein above, these different ink viscosities have different ink-height and ink-penetration in the fabric.

TABLE 3
Line resolution and electrical properties of the printed heaters.

| | | Pressure | Observed | Observed | | Sheet |
|---|---|---|---|---|---|---|
| | | (psi) and | Ink | Ink | Resist- | Resist- |
| Test | Ink | Speed | height | Width | ance | ance |
| Heater | Viscosity | (mm/sec) | (μm) | (mm) | (Ω) | (mΩ/sq) |
| 1 | 0/10 | 42; 70 | 140 | 0.8 | 12 | 5.75 |
| 2 | 1/10 | 6; 70 | 45 | 1.0 | 27 | 16.1 |

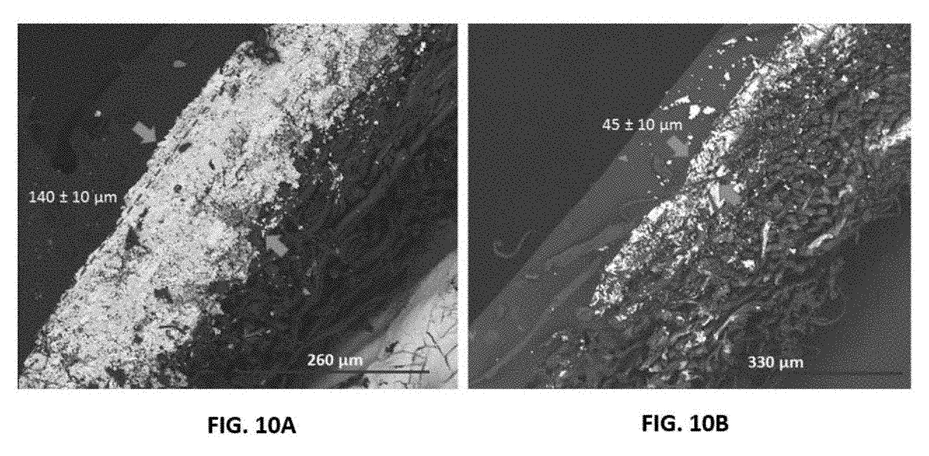

FIGS. 10A and 10B show SEM cross-sectional images of the printed conductive line for Test Heater 1 and Test Heater 2, respectively. As shown in FIG. 10A, there is a higher observed ink height due to the higher viscosity 0/10 and higher fluid pressure (42 psi) depositing more material. In contrast, as shown in FIG. 10B, the fluid pressure (6 psi) and the viscosity 1/10 are both much lower leading to a significantly lower ink height. This flexibility in process variables and material properties can allow for easy fabrication of devices with very different electrical properties.

The data in Table 3 demonstrate the flexibility the disclosed process with easily modified printing parameters (ink viscosity, ink height) that allow facile customization of printed resistive heaters with different line resolution and sheet resistance that are comparable with results obtainable screen-printing [25], but with the improved production characteristics of the disclosed direct-write process. Using the direct-write process, conductive patterns can be printed with much higher ink-height than that of screen-printed structures while keeping similar line resolution that enables ultra-low sheet-resistance of large area printed structures. By varying the ink height of the printed line, we can alter the heating performance of the direct-write printed heaters. This application utilizes the variation of differential ink-height effect on sheet resistance discussed previously.

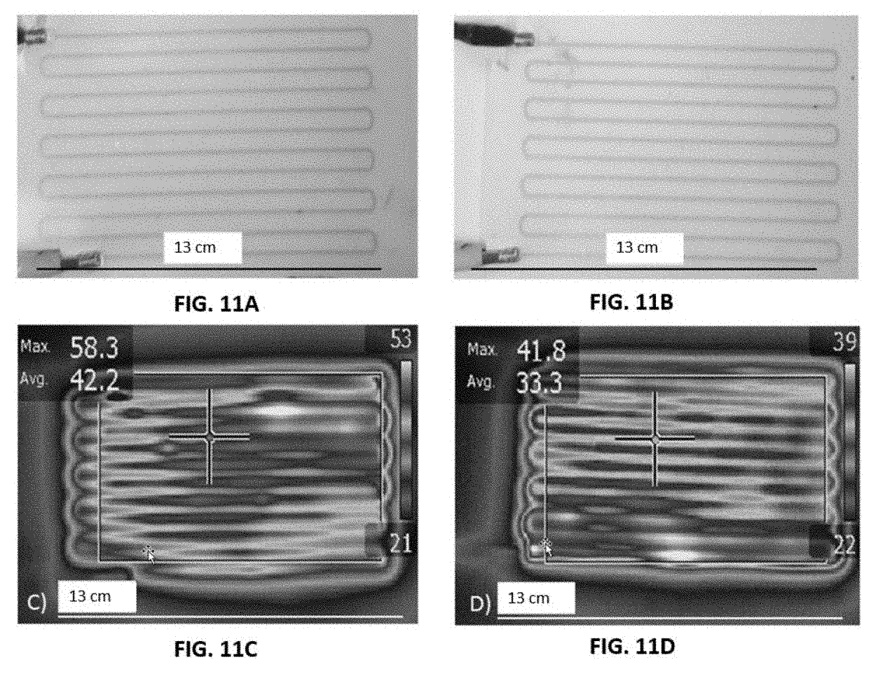

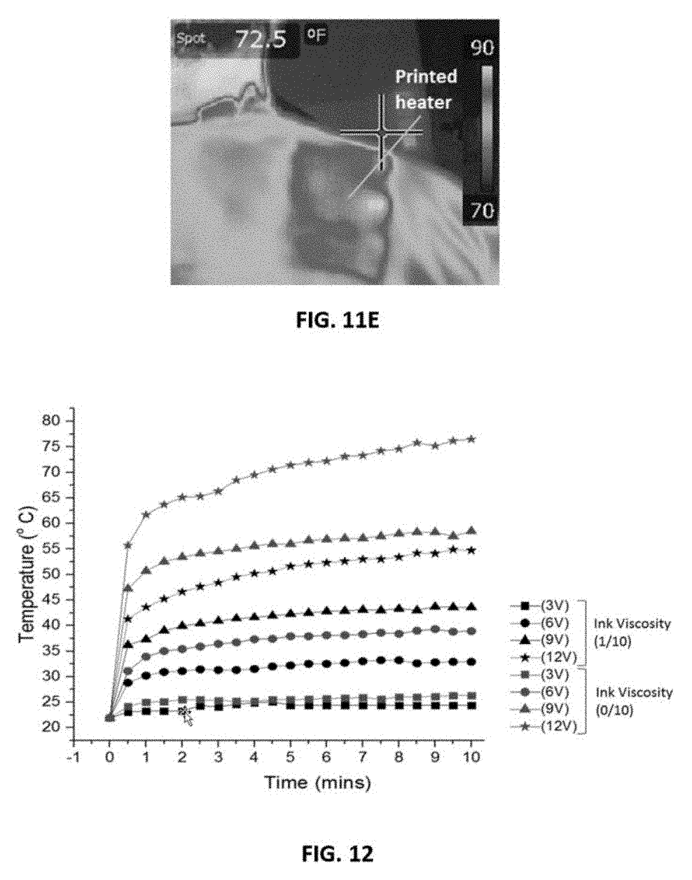

FIGS. 11A and 11B shown an image of Test Heaters 1 and 2, respectively. The corresponding infrared thermal are shown in FIGS. 11C and 11D, respectively. As shown in Table 3, Test Heater 1, which has a lower resistance and a higher ink height, provides a printed resistive heater that is capable of dissipating higher amount of energy in the form of heat than that of Test Heater 2 (compare FIGS. 11C and 11D). FIG. 11E shows an infrared thermal image of a printed resistive heater located in the shoulder area of a shirt garment worn by a human test subject.

The thermal response of the heaters in relation to the differential voltage applied is summarized in FIG. 12. The data show a positive thermal coefficient property of the direct-write printed heaters. For viscosity 0/10, the maximum temperature obtained under the test conditions was around 50° C. as voltage is increased from 3 V to 12 V. As the viscosity decreases, the resistance of the printed heater increases, resulting in an increase in maximum temperature.

Meshed Patch Antenna.

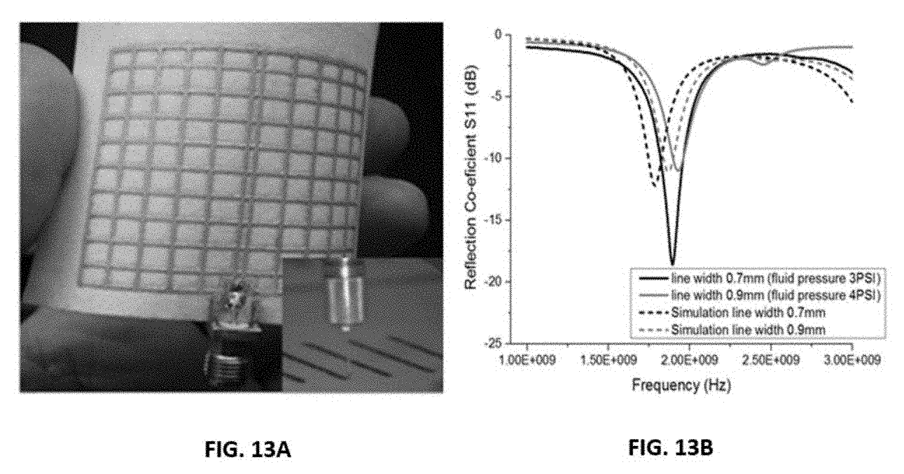

Textile-based communication devices are of great interest in academic research and in industry. Potential uses of textile-based communication devices include wearable antennas or integrated gas filter based antennas for internet-of-things (IoT) applications (e.g., see references [26] and [27]). Facile customization of complex printed antenna designs can be realized by utilizing the disclosed direct-write print processes. In order to test the disclosed direct-write print processes for textile-based communication devices, a meshed patch antenna on Evolon® nonwoven textiles was fabricated using the disclosed methods. The printing was performed using a dispense velocity of 50 mm/sec with a Ag/AgCl ink viscosity of 1/10 as described herein above. Meshed patch antennas were printed using fluid pressures of 3 and 4 psi to yield resulting line widths of 0.9 and 0.7 mm at fluid, respectively. An image of an exemplary meshed patch antenna fabricated by these methods is shown in FIG. 13A. In fabricating the mesh antenna, the fluid pressure can be a critical process metric that influences the line resolution of conductive pattern, which in turn impacts the characteristic impedance and matching of the antenna. For example, the measured reflection co-efficient (S11) of the printed meshed antennas shows that the resonance frequency of the antennas changes with a change in fluid pressure. A full wave electromagnetic simulation (HFSS) compared with the experimental results shows that these printed meshed antennas can be modeled to resonate at a desired bandwidth and frequency range. The direct-write printed mesh antennas on the Evolon® nonwoven is flexible and breathable having ink coverage of only 47.47% compared to traditional patch antennas. The design lends itself to reducing material cost as well as retaining key attributes of the textile substrate. The breathability of the devices provides a unique platform for textile-based sensors that are sensitive to their surrounding environment as well as textile based wearable applications where comfort is of utmost importance. FIG. 13B shows data for the reflection coefficient S11 with respect to the different line-widths compared to simulation data for the same line widths. The data show that an increase in line width results in the shifting of resonance frequency.

3. Flexible 3D-Printed Large Area Resistive Heating Devices

Printing Apparatus.

The studies described in this section used for printing conductive tracks on textiles a modified Nordson Asymtek conformal coating system (Model C-341) using a drop-on-demand mode. The three dimensional movement of nozzle on the Nordson Asymtek conformal coating system is controlled by a robotic hand that has a translation speed up to 508 mm/sec in X-Y axis and 203 mm/s in the Z axis. In order to obtain a high-throughput dispense velocities, for these studies, the needle-to-substrate gap Asymtek conformal coating system was adjusted to a needle-to-substrate gap of about 0.200 to 0.300 mm at a dispense velocity of 40 mm/s and a fluid pressure of 7 Psi. The needle used was a 22 gauge (0.5 mm diameter) needle.

Conductive Ink.

The studies described in this section used a micro-flake based Ag/AgCl ink (Product No. 124-36; Creative Materials Incorporated, Ayer, Massachussetts, USA). The Ag/AgCl ink had Ag:AgCl ratio of 66:34, and a nominal viscosity 10,000 cps (at 1 s⁻¹ shear rate).

Substrate.

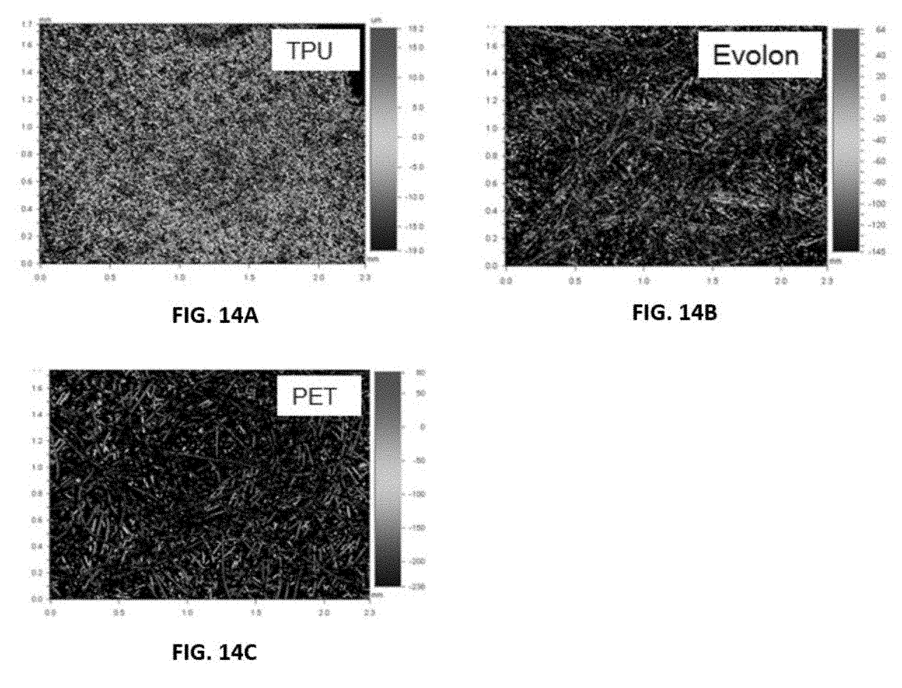

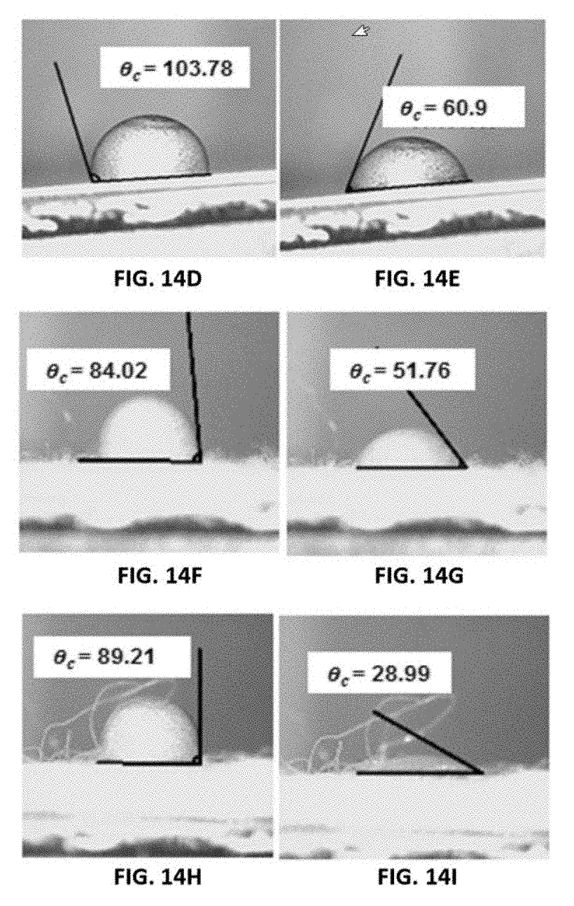

The resistive heating device was fabricated using three flexible substrates: a polyethylene terephthalate (PET) nonwoven fabric, an Evolon® nonwoven fabric (as described herein above), and a thermoplastic polyurethane laminate. The surface roughness and porosity of each substrate was characterized using optical profilometry (Veeco Dektak 150 Profilometer, Veeco Instruments, Inc., Plainview, N.Y.). Representative images are shown in FIGS. 14A-14C. The characteristics of the substrate materials such as roughness, porosity and surface area are given below in Table 4.

TABLE 4
Characteristics of substrates used for fabricating exemplary resistive heater
devices.

| | | Surface | | Surface Area |
|---|---|---|---|---|
| Substrate | | Roughness | | (mm²); scan area |
| Designation | Material | (R_{A}) | Porosity | (mm) |
| PET | Polyethylene terephthalate; | 31.9 μm | 90% | 5.382; (1.7 × 2.3) |
| | nonwoven fabric | | | |
| Evolon ® | Blend of polyethylene | 18 μm | 60% | 8.233; (1.7 × 2.3) |
| | terephthalate and | | | |
| | polyamide; nonwoven fabric | | | |
| TPU-laminate | Thermoplastic polyurethane | 1.5 μm | Not | 3.91; (1.7 × 2.3) |
| | film laminated on polyester | | applicable | |
| | knit fabric | | | |

Fabrication of Printed Resistive Heating Devices.

The resistive heating devices were printed using the Asymtek conformal coating system with the Ag/AgCl conductive ink and the textile substrates as described herein above. Following printing, the printed samples were oven cured at 55° C. for 5 minutes and then heat pressed at 150° C. for 5 minutes. A clear TPU film (Product No. TL 3916; Bemis Company, Inc.; with a nominal thickness of 150 μm) was used to package and encapsulate the printed conductive tracks at 150° C. for device protection and wearability. The printed samples were then connected with copper tape at the printed junction points, i.e., copper film was placed on the conductive ink and then heat-pressed at 1200° C. for 2 minutes. The copper film/conductive ink interface was then encapsulated with silicone for packaging and protection (silicone elastomer; Smooth-On, Inc., Macungie, Pa.). The area of the heaters is chosen 7×13 cm² with a line spacing of 9 mm.

Contact Angle Behavior of the Substrates Tested.

The wetting behavior of the ink on each substrate, the contact angle of each substrate was also characterized immediately after deposition of an ink droplet and then after five minutes on the substrate surface (see FIGS. 14D-14I). The changes of contact angle of the ink droplet after 5 minutes varied significantly with substrate type. The ink droplet on the nonporous TPU laminate spreads within the in-plane direction over time, it spreads both in-plane and through-plane direction on the porous nonwovens, likely due to their 3D structures. It is important to note that the contact angle of the ink decreased significantly on the PET nonwoven compared to Evolon® nonwoven, as shown in FIGS. 14D-14I. The through-plane penetration of the ink in the PET nonwoven is much higher than the Evolon® nonwoven due to the higher porosity and vertical fiber alignment process owed to its needle punching manufacturing process associated with the PET nonwoven fabric.

Characterization of Heating Performance and Durability of Printed Heaters.

The heating performance of the fully packaged devices was characterized by applying 12 V across the device ends from a DC power supply. A FLIR IR camera (FLIR Systems, Inc., Wilsonville, Oreg.) was used to record thermal video images of heating cycles. A single cycle consisted of continuous heating of the device (voltage on) for 5 minutes followed by 5 minutes of cooling (voltage off). Each heating device was tested for 5 consecutive cycles for observing the heating and cooling performance of the devices.

The electromechanical performance of the devices was characterized by using the compression cycling mode of an Instron Mechanical Tester (Instron Engineering Corporation, Norwood, Massachussetts). The flat heating devices were mounted on flat clamps using double sided tape. The clamps of the machine were set to compress 80% of the initial distance between the clamps to fully bend the printed heater devices. The change of the resistance of the heating devices was recorded after every 100 cycles of bending. The durability related to wash/dry cycles, the printed resistive heating devices were washed and dried for 25 cycles following the AATCC 61-2a standard procedure for accelerated washing.

Thermal Response of Printed Resistive Heating Devices.

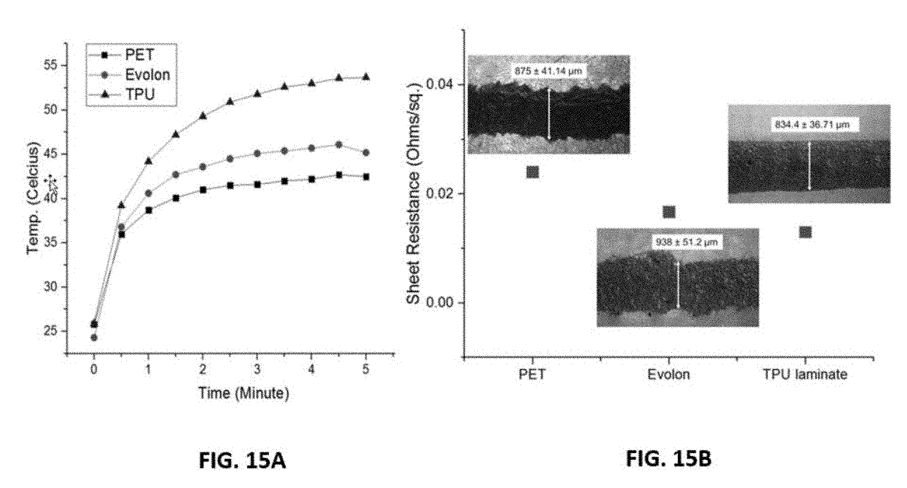

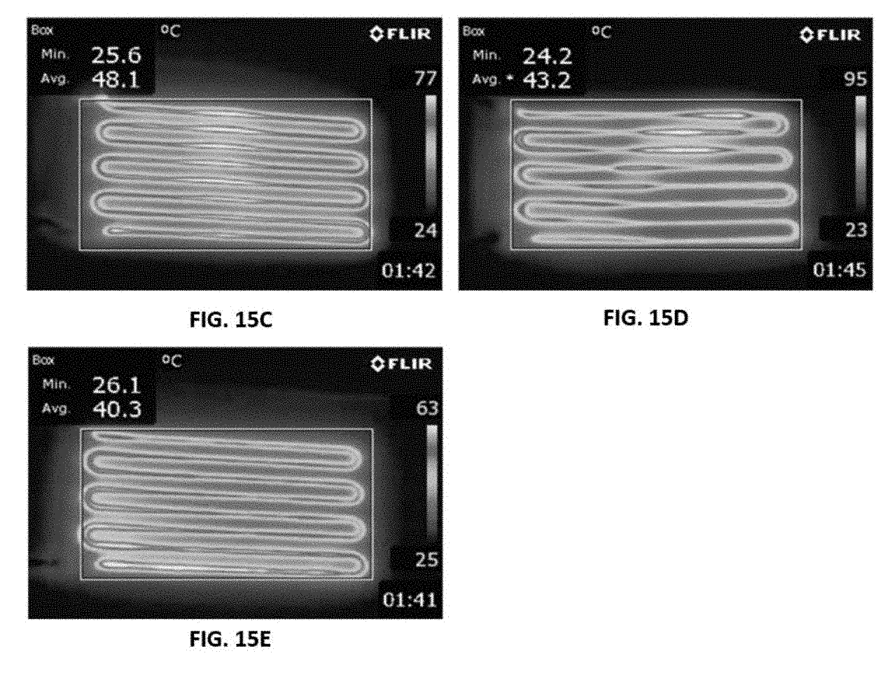

Joule heating is a fundamental property governing the thermal response of printed heaters. The material for used in the devices described herein used a Ag/AgCl conductive ink with micro-flake particles of 65% Ag and 35% AgCl loading by weight percentage (Creative Materials, Ayer, Mass.). Ag has a positive temperature coefficient (PTC), exhibiting an increase in electrical resistance in response to an increase in temperature [41]. This joule heating phenomena is taken advantage of and is fundamentally dependent on the following equation (Equation 4) [40]:

$${T}_{sat} = {T}_{0} + \frac{{U}^{2}}{RhA} , \qquad ( Eq . 4 )$$

where Tₛₐₜ is the saturation temperature after a given length of time, To is the initial temperature, U is the applied voltage, R is the initial resistance, A is the x-sectional area of the conductor, and h is the length of the conductor. From the foregoing equation, it can be seen that as the electrical resistance of the device decreases, the saturation temperature increases. A similar trend was observed with the exemplary devices as shown in our experimental observation as shown in FIG. 15A. The lowest sheet resistance was observed for the printed resistive heater that used the smoothest substrate surface, i.e., the TPU laminate substrate, whereas the highest sheet resistance was observed for the heater printed on the most rough and porous substrate surface, i.e., the PET substrate, shown in FIG. 15B. Infrared thermal images shown in FIGS. 15C, 15D, and 15E the thermal profile for devices printed on TPU laminate, Evolon® nonwoven fabric, and PET nonwoven fabric, respectively. The thermal images were obtained after one minute following the application of voltage in the heating cycle. These thermal images show differences in the average temperature of the printed resistive heaters (cf. FIGS. 15C, 15D, and 15E). Moreover, the thermal images demonstrate the uniformity of heat generation at the conductive lines. In particular, the TPU laminate provided a smooth heated line as the line resolution was better on the TPU laminate substrate, and the ink height was nearly uniform. Without wishing to be bound by a particular theory, the uniformity of the ink height on the TPU laminate substrate could be due to limited porosity of this substrate, thereby limiting ink penetration into the substrate.

SEM Characterization of the Printed Resistive Heating Devices.

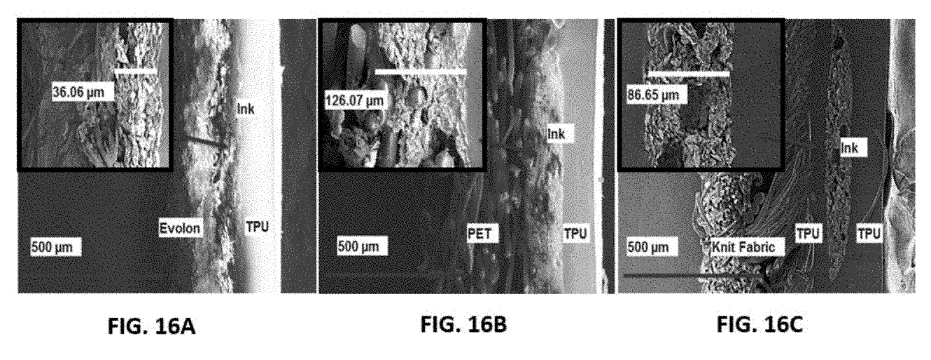

As the data above suggests, the saturation temperature decreased as the surface roughness and porosity of the heaters increased. Without wishing to be bound by a particular theory, it is possible that this primarily results from the morphology of the conductive ink particles within the through-plane direction of the fiber bulk. It should be noted that there is loss in electrical conductivity due to the non-uniformity of the conductive ink layer within the fiber bulk. However, the ink is observed to form a composite with the fibers as seen in SEM images of the cross-section of a printed line on the different substrates (see FIGS. 16A-16C). This unique feature demonstrates the ‘embedded wire’ approach for embedding conductive pathways into the fiber bulk as opposed to on top of the surface of the textile.

The images (FIGS. 16A-16C) show the printed ink height on these different substrates. The penetration of the ink was maximum for the porous PET nonwoven substrate (FIG. 16B), which results in the highest ink height of printed line. In contrast, ink cannot penetrate through the nonporous, smooth TPU laminate substrate (FIG. 16C). Evolon® nonwoven substrate allows ink to penetrate, shows a more limited conductive composite structure of fiber and ink particles (FIG. 16A). Without wishing to be bound by a particular theory, the ability to form a conductive composite structure of fiber and ink particles is limited in the Evolon® nonwoven substrate due to the compact, tight fiber orientation of this material. Of the three substrates tested, the lowest ink height was observed for the printed heater on Evolon® nonwoven substrate, as well as the greatest line width. Without wishing to be bound by a particular theory, the greater line width observed with the Evolon® nonwoven substrate may be due to the compact fibrous structure increasing the capillary flow of the ink in the in-plane direction.

Durability Analysis of Printed Heater.

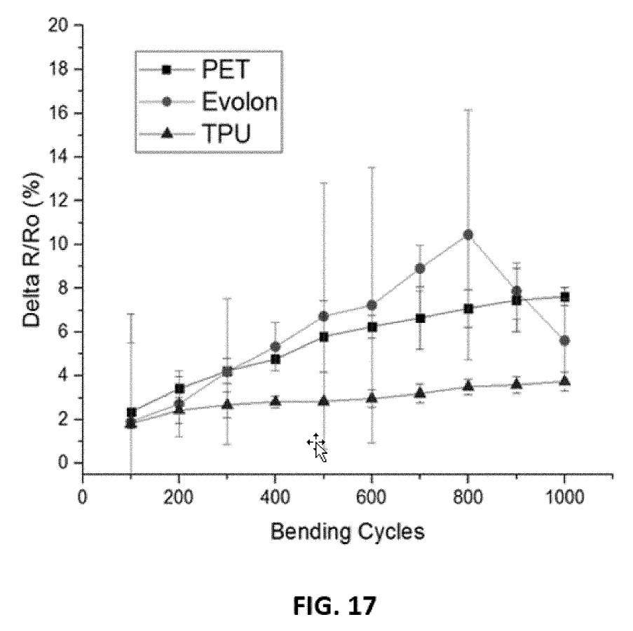

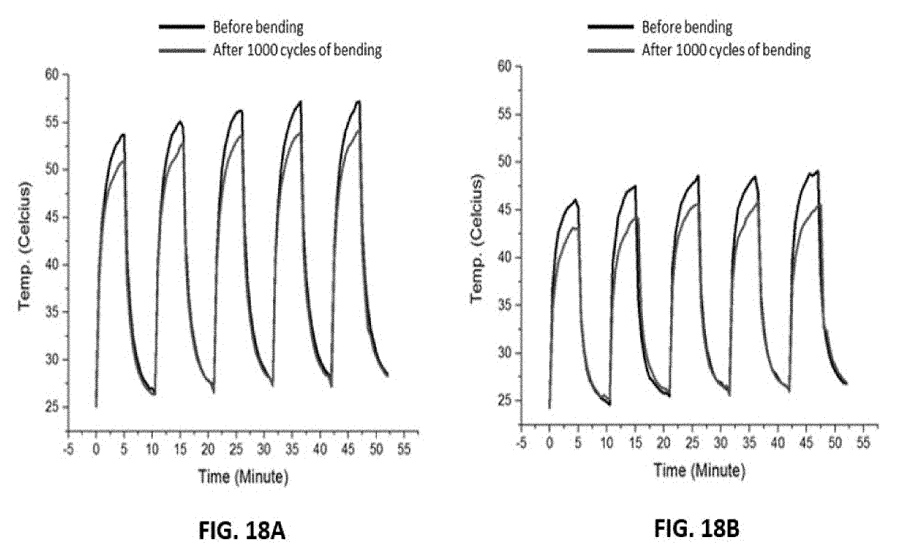

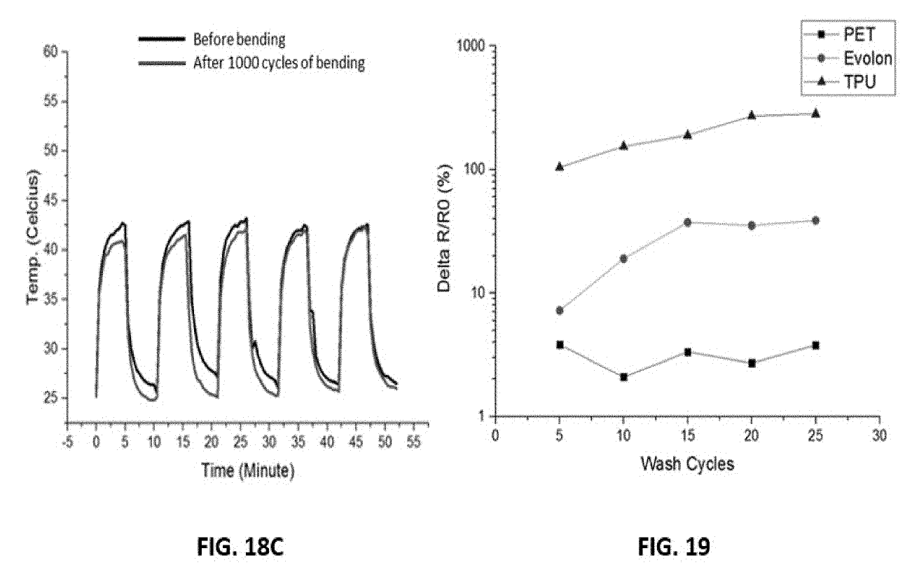

The printed heaters described herein were mechanically flexible with electromechanical stability. The change of resistance of the heaters on TPU laminate, Evolon® and PET nonwoven were 3%, 4%, and 7%, respectively after 1000 cycles of bending (FIG. 17). The data show an interesting tendency of saturation of resistance beyond 800 cycles. The heating-cooling cycles of heaters were also analyzed after 1000 cycles of bending to compare with the native state (FIGS. 18A-18C). In this analysis, each heating cycle consisted of applying DC voltage for 5 minutes on, followed by 5 minutes with the applied DC voltage off. The data show that the maximum temperature achieved during a 5 minute heating cycle was decreased by approximately 2° C. after the device had been subjected to 1000 cycles of bending.

Following the electromechanical characterization and the subsequent analysis of heating performance described above, i.e., following 1000 bending cycles, the printed resistive heating devices were subjected to 25 cycles of wash/dry. The change of resistance was measured after every 5 cycles of wash/dry (FIG. 19). The printed resistive heating devices fabricated on PET nonwoven and Evolon® nonwoven substrates showed an increase of 2.8% and 7% of initial resistance, respectively after 5 cycles of wash/dry. However, the resistance of the printed resistive heating device fabricated on TPU-laminate increased by 100% of initial resistance after 5 cycles of wash/dry. The printed resistive heating device fabricated on Evolon® nonwoven shows a saturation in the change of resistance (12% increase from the initial) after 15 wash/dry cycles and it remains almost the same after 25 wash/dry cycles. In contrast, the heater on PET nonwoven maintains a steady normalized resistance change after 5 wash/dry cycles. It maintains approximately 3% increase from the initial native resistance through 25 wash/dry cycles. Without wishing to be bound by a particular theory, it is possible that the remarkable wash/dry stability of the printed resistive heating device on the PET nonwoven is due to the ink microstructure in the printed lines creating durable conductive composite of fiber and ink particles, thereby enhancing the structural robustness of the conductive material. In contrast, without wishing to be bound by a particular theory, it is possible that the agglomeration of ink structure on the Evolon® and PET laminate substrates yields a less durable structure.

Wearable On-Body Application.

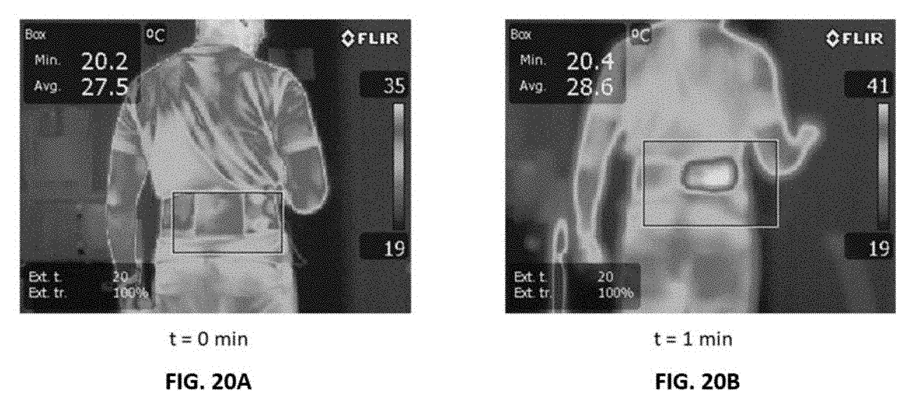

In order to demonstrate the functional efficacy of the exemplary direct-write printed resistive heating device, an on-body demonstration was carried out. Specifically, a printed resistive heating device was fabricated on a PET nonwoven fabric configured as a wearable back wrap for heat therapy. The wearable back wrap was connected to a batter power source (7 VDC/600 mA current). Infrared thermal images show that after 1 minute, the temperature of the heater was about 35° C. (FIGS. 20A and 20B).

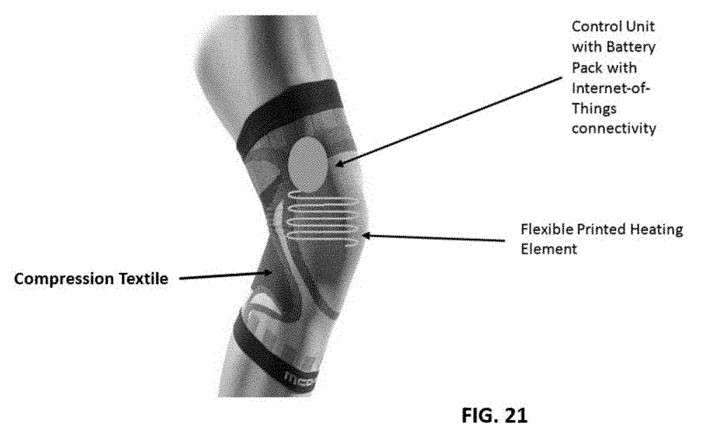

Although, as discussed herein above, the initial sheet resistance of PET nonwoven heater was high due to the ink penetration in the fiber bulk, the printed resistive heating device using this textile substrate showed superior durability performance compared to the printed resistive heating devices using either the Evolon® nonwoven or the TPU laminate substrates. The data disclosed herein suggest that the embedded ‘wire-like’ structure of ink and fibers in the printed line on a PET nonwoven substrate appears to maintain the electrical conductive bridge after extreme mechanical deformation and washing processes. Thus, the disclosed methods utilizing a PET nonwoven substrate, or other textile with similar properties, can be used to fabricate wearable textile electronic devices for the healthcare and wearable technology markets. An exemplary device for the healthcare market is shown in FIG. 21 comprising a compression textile, comprising a PET nonwoven substrate, further comprising a printed resistive heating device and a portable power source, such as a battery.

4. Printed Vertical Interconnect Access (VIA) of Flexible Circuit Board on Nonwoven Fabrics

Using the disclosed direct-write methods, which provide facile and high-speed fabrication processes, an exemplary vertical interconnect access (VIA) on textile platform was fabricated. The VIAs were printed with conductive silver paste on a needle-punched polyester (NPPET) nonwoven fabric using disclosed direct-write printing processes. Fully printed patterns of silver conductive tracks were connected by VIAs on the both side of the NPPET nonwoven fabric. Additionally, the durable connected VIAs were printed on thick-laminated NPPET nonwoven of 1.2 mm. The ink morphology of the VIAs showed a composite microstructure of silver flakes and fibers, which impart the mechanical robustness and conductive electrical network.

Needle-punched polyethylene terephthalate (NPPET) nonwoven fabric was utilized as a substrate on which to print conductive patterns connected with VIAs. The NPPET nonwoven fabric layers were heat-laminated with a porous thermoplastic polyurethane (TPU) web. The TPU web works as an adhesive layer without changing the porous structure of the nonwoven fabrics. Three different fabric samples were prepared by the heat-press process (at 150° C.). The heat-press process aids in smoothing the fabric surface roughness which facilitates printing conductive lines with good surface resolution. Table 5 below shows characteristics for the surface profile of the NPPET nonwoven laminated fabrics used in these studies.

TABLE 5
Surface profile of needle-punched PET nonwoven laminated fabrics

| | | | | Surface area |
|---|---|---|---|---|
| Sample | No. of | | Surface | (mm²); scan |
| No. | layers | Thickness | roughness (Rₐ) | area 3.91 mm² |
| 1 | 1 layer | 0.35 ± 0.02 mm | 32 μm | 5 mm² |
| 2 | 2 layers | 0.9 ± 0.08 mm | 32 μm | 5 mm² |
| 3 | 3 layers | 1.2 ± 0.1 mm | 32 μm | 5 mm² |

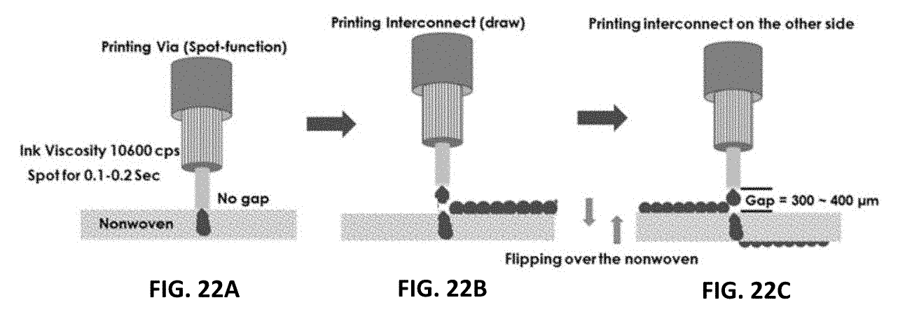

An Asymtek C-341 conformal coating machine (as described herein above) was modified to carry-out drop-on-demand direct-write printing with conductive ink. The conductive ink was Ag/AgCl ink with viscosity 10,000 cp (at 1 s-1 shear rate), i.e., corresponding to the viscosity 1/10 as described herein above (Creative Materials). The conductive pattern was drawn on both sides of the nonwoven fabric as shown in FIGS. 22A-22C. The printing process was carried out in two separate steps. First, the VIAs are printed on the pre-determined via points (FIG. 22A). During this process, the nozzle comes very close to the fabric surface and dispense ink on the single point for 0.1-0.2 seconds. The capillary flow of the ink (fluid) draws the ink in the vertical direction. The fiber spacing, along with the porosity of the NPPET nonwoven fabric, was decreased due to the heat-press process. Without wishing to be bound by a particular theory, as the fiber spacing decreased, the capillary force likely increased due to the increase of surface area. However, the pore size of the heat-pressed nonwoven fabric was, nevertheless, apparently large enough to allow flow of the ink in the vertical direction. Dispensing ink for 0.2 seconds appeared to be sufficient to create a VIA through a 1.2 mm thick NPPET nonwoven fabric.

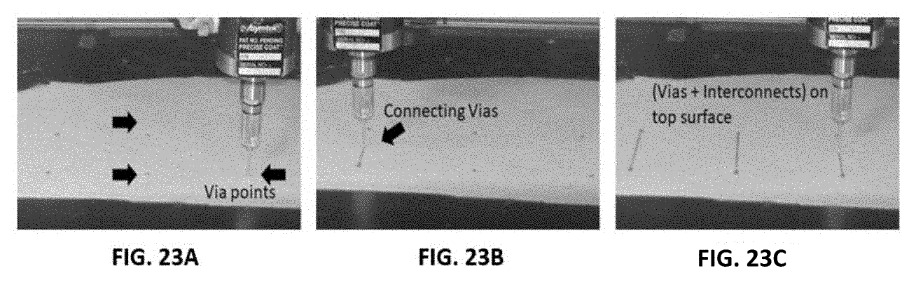

After printing VIAs in the designated spots, interconnects were printed on both sides of the nonwoven fabric (FIGS. 22B-22C). During printing interconnects, the gap between the dispenser needle and the fabric was maintained at about 300-400 μm. Maintaining this gap was important for printing continuous conductive line on the relatively rough surface of the NPPET nonwoven fabric. In the absence of an appropriate gap between the dispenser needle and the fabric, the uneven, rough surface and the protruding fibers on the fabric can obstruct the continuous ink ejection during printing. The process of printed VIAs and connected interconnect lines is further shown in FIGS. 23A-23C.

After printing VIAs and the connected interconnect lines, the printed pattern was heat-pressed and encapsulated with the porous TPU web at 120° C. for 5 minutes. The thin (0.1 mm) TPU web provides wear protection for the printed pattern, e.g., mitigating erosion or abrading of the printed conductive that can arise from rough handling and mechanical deformation. Although the encapsulating TPU web provides suitable wear protection, it does not appear to impact flexibility of the printed device.

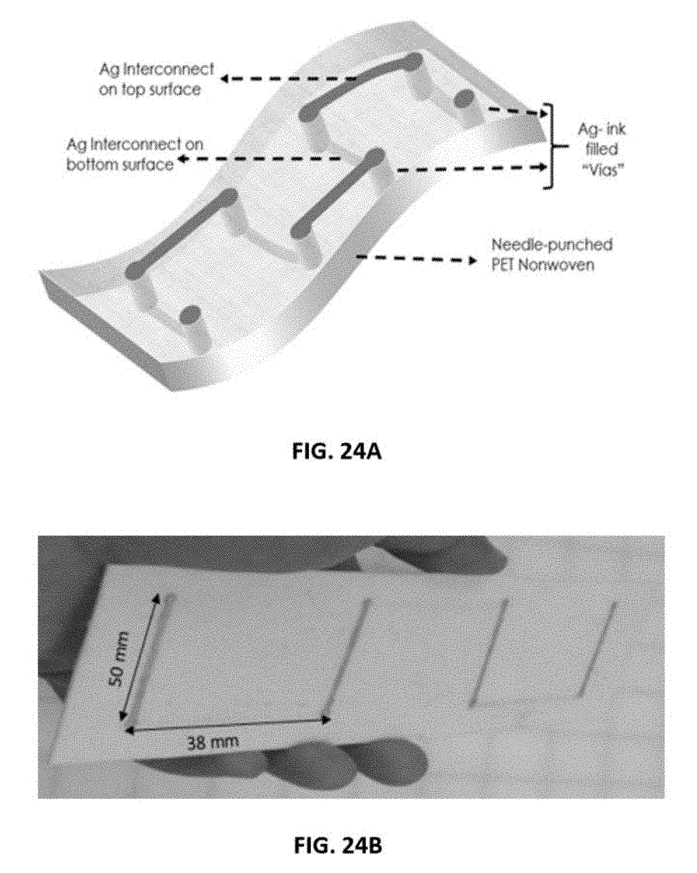

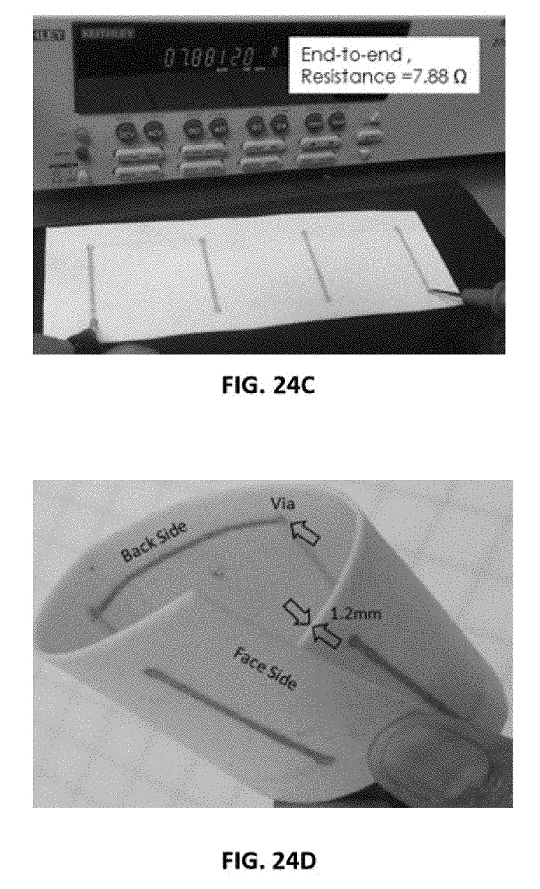

A schematic representation of a device comprising VIAs and interconnects is shown in FIG. 24A. An image of an exemplary device printed on 1.2 mm thick NPPET nonwoven fabric substrate is shown in FIG. 24B. As shown in FIG. 24C shows that the resistance of the end-to-end points of the printed pattern was 7.88Ω, thus demonstrating that the fully printed pattern was electrically conductive and that the VIAs were able to connect the printed interconnects vertically from both sides of the nonwoven substrate. FIG. 24D shows that the printed VIA pattern on 1.2 mm thick NPPET nonwoven fabric substrate is flexible.

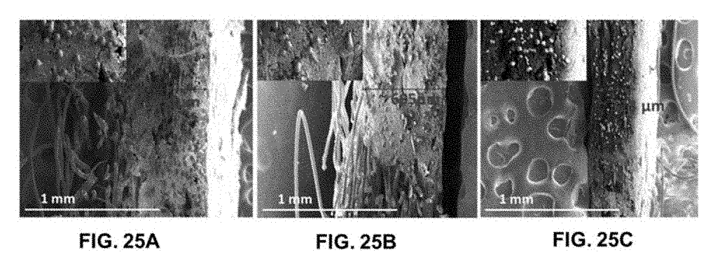

SEM imaging was carried on a cross-sectional view of the along the direction of printed VIAs to assess the ink-microstructure in the fiber mat. The images (FIGS. 25A-25C) show that the conductive ink penetrated in the fiber bulk and created a composite, electrically connected network vertically through the fiber bulk.

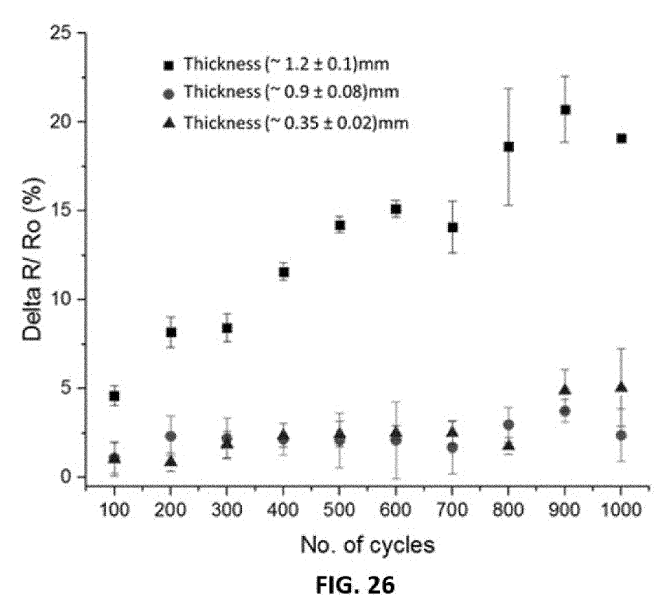

The reliability and the robustness of the printed VIA patterns under repeated mechanical deformation was determined. FIG. 26 shows the change of electrical resistance of the printed VIA patterns after 1000 cycles of bending. The printed VIAs with the thickness of 0.35 mm and 0.9 mm realized an increase of resistance to only 5% from the initial values after 1000 cycles of bending. The resistance increased about 20% from the initial value after 1000 cycles of bending for the printed VIA networks with the thickness of 1.2 mm. Without wishing to be bound by a particular theory, there is a higher chance of dislocating the conductive particles in the thicker VIAs. However, all the circuits with VIAs on each thickness of NPPET nonwoven fabric tested showed an acceptable range of resistance increase after the repeated cycles of mechanical deformation. Without wishing to be bound by a particular theory, it is possible that the composite structure of the ink and fiber matrices enabled the robust durability, flexibility and electrical properties of the printed VIAs.

The VIAs fabricated and analyzed herein above demonstrate that the disclosed methods provide a facile process of 3D printing conductive VIAs in the nonwoven textile materials with thickness up to 1.2 mm, and that the VIAs have robust durability and flexibility. In various aspects, integration of such types of VIAs in a fabric circuit will facilitate the implementation of flexible and durable printed circuit board (PCB) for wearable E-textile applications.

5. Prospective Multilayer Textile Heating Device

A prospective design is provided herein for a wearable multilayer resistive heating textile package comprising a resistive heater packaged in a multilayer design providing improved comfort and breathability. The multilayer textile heating device can be configured as a heating back-wrap to provide pain-relief. However, the design and construction aspects are versatile, and can be utilized in other articles, such a heating jacket, heating gloves, heating furniture and the like, without departing from the scope or spirit of the disclosed multilayer textile heating device.

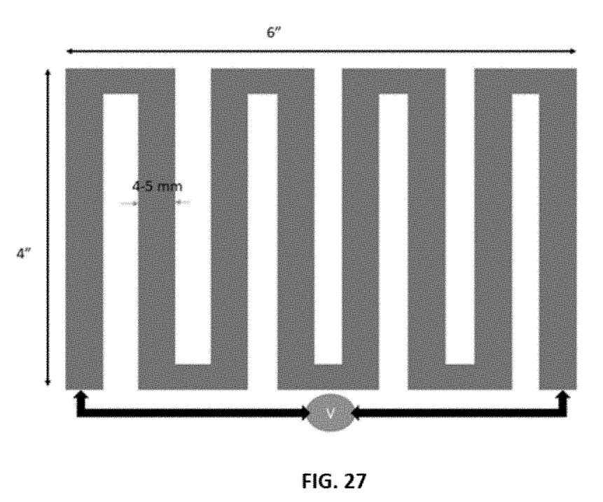

FIG. 27 shows a schematic representation of a resistive heating device. The representative device has dimensions of about 4″×6″, with conductive lines with a line width of about 4-5 mm. The conductive lines can be fabricated from conductive ink using the disclosed direct-write printing methods or screen-printed using conductive inks. Alternatively, the conductive lines can be cut in the indicated pattern from a conductive fabric material. As shown in the figure, the conductive lines interface to a portable DC power source. The conductive fabric can be plated with Ni or Cu, and have a sheet resistance of 0.03 Ω/sq. The resistance of the resistive heating device is about 5-6Ω. The resistive heating device shown in FIG. 27 comprises a VDC power source (indicated by the component labeled “V” in the figure). In some aspects, the power source is one or more non-rechargeable battery. Alternatively, in other aspects, the power source is one or more non-rechargeable battery. The power source is configured to provide about 9-12 VDC. The geometry and dimensions of the resistive heating device heater shown in FIG. 27 is anticipated to decan generate about 110° F. upon applying 9 V across the ends.

The active heating area of the heater is very important to identify to design a product. It is noteworthy to mention that in order to generate adequate amount of heat; the resistance of the heater should be low enough and the value of that resistance can be determined by the following equation (Equation 5):

$${T}_{sat} = {T}_{0} + \frac{{U}^{2}}{RhA} . \qquad ( Eq . 5 )$$

where Tₛₐₜ=the saturation temp., T₌₀=the initial temp., U=input voltage, R=resistance of the heater, A=surface area of the heater, h=heat transfer co-efficient (which is dependent upon the composition of the packaging material).

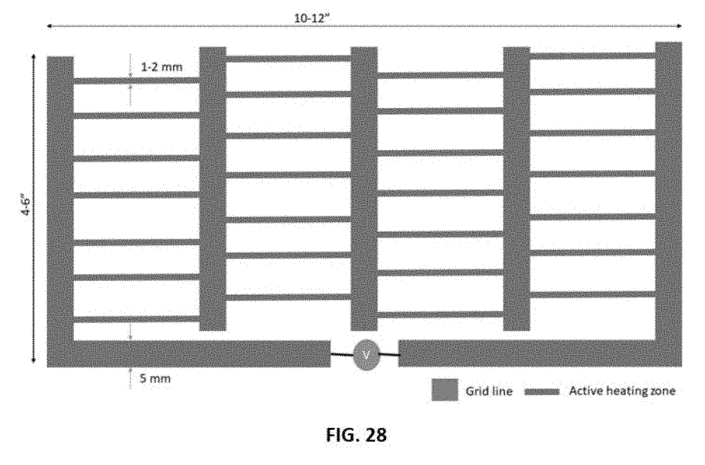

The resistive heating device can be designed to provide an active heating area over a larger area without loss of desired heating levels. For example, the active heating zone can be distributed with a grid-like heating element as shown in FIG. 28. The representative device shown in FIG. 28 has dimension of about 4-5″×10-12 inches. Accordingly, this device encompasses about 40-60 in², compared to about 24 in² for the device shown in FIG. 27. In order to optimize performance for a device covering the larger area, as shown in FIG. 28, a distributed grid system is utilized. Briefly, the device comprises a network of lower resistance conductive lines (i.e., the conductive lines shown as having a line width of about 5 mm), which are interconnected with higher resistance conductive lines (i.e., the conductive lines shown as having a line width of about 1-2 mm). The resistive heating device would generate resistive heat at the higher resistance interconnecting conductive lines, whereas the lower resistance conductive lines would generate almost no resistive heat by comparison due to the lower resistance of these lines. The resistive heating device shown in FIG. 28 comprises a VDC power source (indicated by the component labeled “V” in the figure). In some aspects, the power source is one or more non-rechargeable battery. Alternatively, in other aspects, the power source is one or more non-rechargeable battery. The power source is configured to provide about 9-12 VDC.

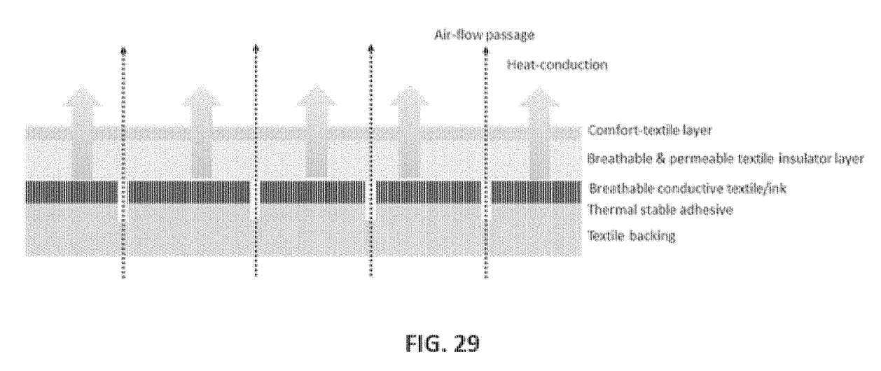

The wearable multilayer resistive heating textile package comprises a resistive heater packaged in a multilayer design providing improved comfort and breathability. A cross-sectional view of the multilayer structure is shown in FIG. 29. The layer indicated as the breathable conductive textile/ink layer can comprise any resistive heating device disclosed herein, including those devices depicted in FIGS. 27 and 28. As shown in FIG. 29, the breathable conductive textile/ink layer is disposed in the multilayer structure comprises a first layer comprising a textile backing, a second layer comprising a thermal stable adhesive, a third layer comprising the breathable conductive textile/ink layer, a forth layer comprising a permeable textile insulator, and a fifth layer comprising a comfort-textile layer. The first layer provides a backing for the resistive heating device, which as described herein can be printed using conductive inks using a disclosed direct-write printing method or printed using a silk screen printing method, or alternatively is a conductive fabric cut to the desired pattern, such as a meander line pattern, and is the glued or sewn onto the first layer. Integration of a thermally stable adhesive can improve the durability of the heating materials. A breathable and thermally conductive textile layer, i.e., the fourth layer as shown in FIG. 29, can been layered onto the heating material by simple cut and sew process. The fourth layer facilitates evenly distributing heat over the heating area. Finally, a fifth layer, comprising a thin textile comfort layer provides comfort and good heat conduction to the human body.

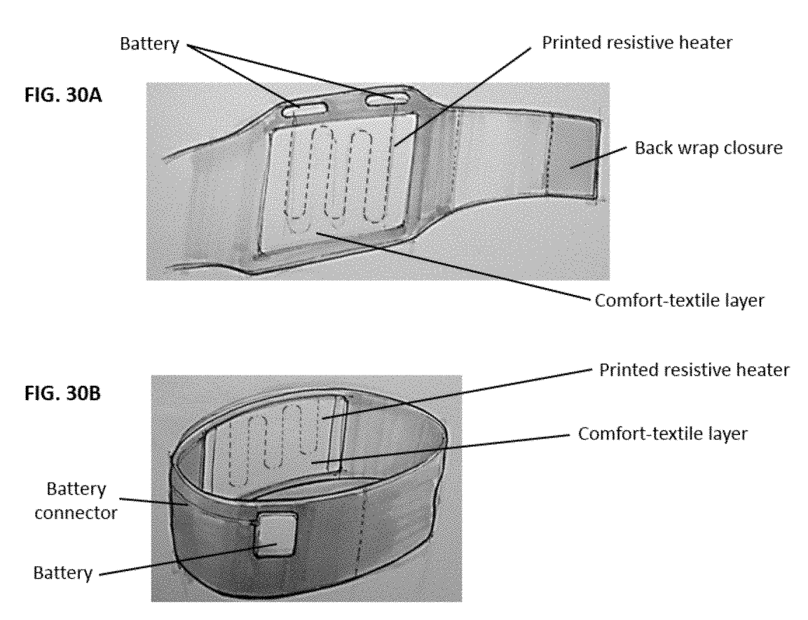

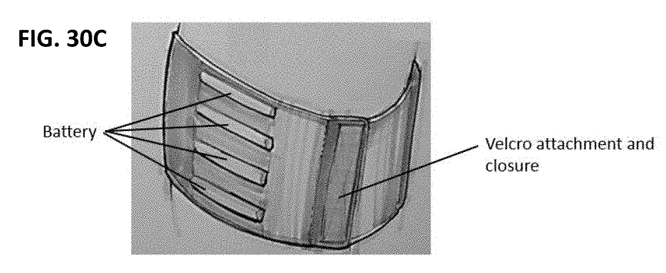

The multilayer resistive heating textile package can be further incorporated into a variety of articles, such as the wearable heating back wrap shown in FIGS. 30A-30C. FIGS. 30A-30C each show a representative wearable heating back wrap. Highlighted in each of FIGS. 30A-30C are particular aspects of the given wearable heating back wrap. FIG. 30A shows one configuration of a wearable heating back wrap that can be secured about a torso via the back wrap closure, which can comprise a hook-and-loop closure, adhesive tape, or a strap and buckle design. FIG. 30B shows an alternative version of a wearable heating back wrap that is formed using a fabric comprising a stretchable fabric such as a spandex and that forms a continuous garment that can be secured about a torso due to the tension provide by the stretchable fabric. FIG. 30C shows a further alternative wearable heating back wrap configured with a series of batteries oriented approximately perpendicular to the spine, such that the wearable heating back wrap provides for bending mobility when engaged on a torso. The wearable heating back wrap shown in FIG. 30C further comprises a closure system comprising a hook-and-loop attachment.

The disclosed methods provide the fundamental materials-process relationships required for printing conductive ink structures onto textile substrates by means of a direct-write process. The disclosed methods provide optimized fluid pressure, dispense velocity, and ink viscosity that can be utilized for novel large-area textile electronics. For applications of printing technologies onto textiles, commercially viable processes require: (a) a high-throughput printing method that allows for control of dispense velocity and fluid pressure of ink; (b) a textile substrate with high liquid absorbency to absorb solvent and leave metallic ink percolation intact; and (c) a low-cost conductive ink. These requirements are met by the disclosed methods that provide a drop-on-demand mode of ink deposition system, suitable fluid pressure ranges according to the viscoeleatic behavior of the ink, and selection of a suitable textile substrate characterized by a low surface roughness with high surface area. The disclosed methods allowed printing of conductive tracks at a dispense velocities that were about 8-fold greater than previously described methods. Moreover, as described herein, the disclosed methods provide ink deposition that can be controlled in three-dimensions, thus allowing for controlled variation of the performance of the printed devices such as interconnects, heaters, and antennas.

The disclosed methods, when used to print a conductive ink on a textile, provide a route embedded ‘wire-like’ composite structure made of fibers and conductive ink. The disclosed products produced using these methods show significant durability when subjected to repeated mechanical stresses that would be encountered during normal wearability and washing. Accordingly, the disclosed direct-write methods can be utilized to fabricate products such as smart heated garments such as socks, underwear, shirts, pants, and jackets.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A method of forming a conductive material comprising applying a conductive ink with a printer to a substrate material;
wherein the conductive ink comprises a conductive microparticle;
wherein the conductive ink comprises a polymer binder;
wherein the conductive ink comprises a solvent suspensions system;
wherein the conductive ink has a viscosity of about 10,000 cps to about 100,000 or more cps when determined at a 1 s⁻¹ shear rate;
wherein the printer comprises a drop on demand ink jet printhead comprising at least one nozzle;
wherein the at least one nozzle tip is at a distance of about 0.1 mm to about 0.4 mm from the substrate material;
wherein the conductive ink is dispensed from the at least one nozzle at a dispensing velocity of about 50 mm/s to about 200 mm/s; and
wherein the conductive ink is dispensed from the at least one nozzle at a fluid pressure of about 1 psi to about 100 psi.

2. The method of claim 1, wherein the conductive microparticle comprises one or more elements each selected from the group consisting of: an element from Group 3 to Group 14 of the Periodic Table of Elements, one or more conductive polymers, and combinations thereof.

3. The method of claim 2, wherein the one or more elements is selected from the group consisting of: silver, copper, gold, nickel, aluminum, or combinations thereof.

4. The method of claim 2, wherein the one or more elements is from Group 14 and is selected from the group consisting of: carbon, tin, silicone, and combinations thereof.

5. The method of claim 2, wherein the conductive polymer is selected from the group consisting of: a poly(fluorene), a polyphenylene, a polypyrene, a polyazulene, a polynaphthalene, a polyacetylene, a poly(p-phenylene vinylene), a poly(pyrrole), a polycarbazole, a polyindole, a polyazepine, a polyaniline, a poly(thiophene), a poly(3,4-ethylenedioxythiophene), a poly(p-phenylene sulfide), and combinations thereof.

6. The method of claim 1, wherein the conductive microparticle comprises a combination of silver and silver chloride; and wherein the silver and silver chloride are present in a weight ratio of about 50:50 to about 75:25.

7. The method of claim 1, further comprising applying a dielectric ink in combination with the conductive ink.

8. The method of claim 1, wherein the substrate material is a textile selected from the group consisting of: a woven fabric, a knit fabric, a composite fabric, a nonwoven fabric, and combinations thereof.

9. The method of claim 8, wherein the textile has a surface roughness (R_{A}) of about 10 μm to about 40 μm.

10. The method of claim 8, wherein the textile comprises a fiber or filament comprising cotton, cellulose, a combination of cotton and cellulose, polyethylene terephthalate, polyamide, polyester, thermoplastic polyurethane, or combinations thereof.

11. The method of claim 8, wherein the textile has a porosity of about 40% to about 99%.

12. The method of claim 1, wherein the substrate material is a film.

13. The method of claim 12, wherein the film comprises polyethylene terephthalate, polyamide, polyester, thermoplastic polyurethane, or combinations thereof.

14. The method of claim 1, further comprising curing the conductive material and substrate material after applying the conductive ink to the substrate material.

15. The method of claim 14, wherein curing comprises heating the conductive material and substrate material at a temperature of about 25° C. to about 150° C. for a period of about 1 minute to about 30 minutes and wherein curing is conducted using a technique selected from the group consisting of: a radiation based curing process, a thermal based curing process, and combinations thereof.

16. The method of claim 1, further comprising encapsulating a surface of the conductive material with a thermoplastic elastomer.

17. The method of claim 1, further comprising forming a vertical interconnect access, comprising applying a conductive ink with a printer to a substrate material;
wherein the at least one nozzle tip is placed at a single point of contact on a first surface of the substrate material;
wherein the conductive ink is dispensed from the at least one nozzle at the single point of contact for a contact time of about 0.05 seconds to about 0.5 seconds; and
wherein the nozzle tip to fabric surface gap is essentially zero;
thereby forming the vertical interconnect access.

18. An article comprising a component made by the method of claim 1.

19. The article of claim 18, wherein the component is a printed resistive heating device, a printed antenna, a vertical interconnect access, a sensor, or combinations thereof.

20. The article of claim 18, wherein the article is a garment, an article of apparel, an article of footwear, an article of protective clothing, a helmet, a hat, a sock, a glove, a ballistic material, or an article of body armor, a medical device, a wound covering, a wound dressing, a medical mesh, a medical fabric, or an orthopedic support device.
